(19) United States
(12) Patent Application Publication
(10) Pub. No.: US 2026/0055375 A1
(43) Pub. Date: Feb. 26, 2026

(54) CULTURE METHOD

(71) Applicant: Babraham Institute, Cambridge (GB)

(72) Inventors: Matteo MOLÉ, Cambridge (GB); Peter RUGG-GUNN, Cambridge (GB)

(21) Appl. No.: 19/316,794

(22) Filed: Sep. 2, 2025

Related U.S. Application Data
(63) Continuation of application No. PCT/GB2024/050559, filed on Mar. 1, 2024.

(30) Foreign Application Priority Data
Mar. 3, 2023 (GB) 2303138.8

(51) Int. Cl. C12N 5/071; C12N 5/073

(52) U.S. Cl. CPC C12N 5/0682; C12N 5/0604; C12N 2500/32; C12N 2500/38; C12N 2501/01; C12N 2501/11; C12N 2501/119; C12N 2501/12; C12N 2501/155; C12N 2501/392; C12N 2501/415; C12N 2501/727; C12N 2503/04; C12N 2513/00; C12N 2533/52; C12N 2533/54; C12N 2533/70

(57) ABSTRACT

The invention relates to a method of generating an in vitro endometrium assembloid comprising growing endometrial stromal cells inside a hydrogel matrix and layering cells and/or fragments from endometrial epithelial organoids on top, thereby generating a three-dimensional endometrium assembloid comprising a stromal compartment, an outer luminal epithelial layer and a functional secretory glandular endometrium. The invention further relates to an in vitro endometrium assembloid produced according to the methods defined herein. Also provided are methods for determining embryo implantation ability and/or window of receptibility to embryo implantation and for screening for embryo implantation-modulating agents comprising, providing the in vitro endometrium assembloid defined herein. The invention further relates to the use of three sequential human embryo media (HEM) for culturing human stem cell based embryo-like (blastoids) or human blastocysts, and to the compositions of the three media.

This application is a bypass continuation of International Application No. PCT/GB2024/050559, filed Mar. 1, 2024, which claims the benefit of priority of United Kingdom Application No. 2303138.8, filed Mar. 3, 2023, the contents of which are incorporated by reference as if written herein in their entireties.

### FIELD OF THE INVENTION

The invention relates to a method of generating an in vitro endometrium assembloid comprising growing endometrial stromal cells inside a hydrogel matrix and layering cells and/or fragments from endometrial epithelial organoids on top, thereby generating a three-dimensional endometrium assembloid comprising a stromal compartment, an outer luminal epithelial layer and a functional secretory glandular endometrium. The invention further relates to an in vitro endometrium assembloid produced according to the methods defined herein. Also provided are methods for determining embryo implantation ability and/or window of receptibility to embryo implantation and for screening for embryo implantation-modulating agents comprising, providing the in vitro endometrium assembloid defined herein. The invention further relates to the use of three sequential human embryo media (HEM) for culturing human stem cell based embryo-like (blastoids) or human blastocysts, and to the compositions of the three media.

### BACKGROUND OF THE INVENTION

Implantation is a key stage of human embryogenesis, whereby physical connections between the developing embryo and the maternal uterus are established for the very first time. In a narrow time period, at the transition between the first and second week of gestation known as the window of implantation, the human embryo implants into the maternal womb (Wilcox, Baird & Weinberg (1999) N. Engl. J. Med., 340(23):1796-1799, doi: https://doi.org/10.1056/nejm199906103402304). This represents the earliest event underlying the establishment of a successful pregnancy. The foetal-maternal interactions initiated at this stage lay the foundation for the formation of the placenta, supporting pregnancy progression and sustaining intrauterine gestation of the conceptus until birth.

This event is particularly susceptible to failure. It is estimated that 30-60% of human embryos are unable to undergo successful implantation, resulting in spontaneous pre-clinical miscarriages (Wilcox, Baird & Weinberg (1999); Wilcox et al. (1988) N. Engl. J. Med., 319(4):189-194, doi: https://doi.org/10.1056/nejm198807283190401; and Koot et al. (2012) Biochim Biophys Acta, 1822(12):1943-1950, doi: https://doi.org/10.1016/j.bbadis.2012.05.017). The high incidence of implantation failure is estimated to be one of the major limiting factors behind the low efficiency of in vitro fertilisation (IVF) procedures. Despite the advancement in embryo quality selection and pre-implantation genetic diagnosis in fertility clinics, a significant proportion of embryos still fail at this stage. Due to this ‘implantation barrier’, patients are often required to undergo several cycles of IVF treatment, with chances of live births ranging from a maximum of 32% success down to 2%, depending on age and health conditions of the patient (Human Fertilisation and Embryology Authority [HFEA], Fertility treatment 2017: Trends and Figures Report (2019)).

Despite the significant clinical impact, implantation is commonly referred as a ‘black box’ of human development (Macklon, Geraedts, & Fauser (2002) Hum. Reprod. Update, 8:333-343, doi: https://doi.org/10.1093/humupd/8.4.333), because of the very limited knowledge and lack of suitable model systems to study this stage. Studying this process has been a significant challenge due to the inaccessibility to directly observe implantation in vivo, since the embryo becomes progressively hidden and burrowed inside the endometrium. Most, if not all, of our knowledge of this stage derives from histological examinations of hysterectomies conserved at the Carnegie Institution in USA (Hertig, Rock & Adams (1956) Am. J. Anat., 98:435-493, doi: https://doi.org/10.1002/aja.1000980306; Hertig, & Rock (1949) Contrib. Embryol., 33:169-186, PMID: 18130387; Hertig, & Rock (1945) Contrib. to Embryol., 31; Hertig, & Rock (1941) Carnegie Institution of Washington; and Heuser, Rock & Hertig (1945) Contrib. Embryol., Carnegie Inst. Wash. Publ., 557:85-99) and in the Boyd Collection in UK (Benirschke (1973) Teratology, 8:77-78, doi: https://doi.org/10.1002/tera.1420080118). These specimens have provided invaluable insights into the cytoarchitecture of human implantation in vivo, serving as the foundation of what we know today about this stage. However, the scarcity of available specimens and limitation to histological examination prevent deep understanding of the molecular and cellular mechanisms underlying this critical stage of human development.

Advances in culture conditions have enabled the extension of survival of human embryos beyond 5-6 days post-fertilisation (d.p.f.; Deglincerti et al. (2016) Nature, 533:251, doi: https://doi.org/10.1038/nature17948; Shahbazi et al. (2016) Nat. Cell Biol., 18:700-708, doi: https://doi.org/10.1038/ncb3347; and Xiang et al. (2020) Nature, 577:537-542, doi: https://doi.org/10.1038/s41586-019-1875-y). However, because of the absence of uterine tissue, embryos are unable to undergo implantation and develop outside their physiological environment. This has hindered the possibility to recapitulate the process of human embryo implantation in vitro and to explore the complex interactions established between the embryo and the maternal endometrium at this critical stage.

There is therefore a need to develop a system which recreates the functional endometrial environment and allows the implantation of a blastoid or blastocyst in vitro, opening opportunities to investigate and study the complex process of human implantation. Such systems may also allow the determining of the embryo implantation ability and/or window of receptibility to embryo implantation for an individual, as well as the screening for agents which have embryo implantation-modulating effects. These agents may, in turn, be used to enhance implantation ability or hinder implantation, and thus be used as contraception.

### SUMMARY OF THE INVENTION

According to a first aspect of the invention, there is provided a method of generating an in vitro endometrium assembloid, said method comprising the steps of:
(i) growing endometrial stromal cells inside a hydrogel matrix; and
(ii) layering cells and/or fragments from endometrial epithelial organoids on top of the cultured endometrial stromal cells in hydrogel matrix of step (i);
thereby generating a three-dimensional endometrium assembloid comprising a stromal compartment, an outer luminal epithelial layer and a functional secretory glandular endometrium,
wherein the hydrogel matrix comprises one or more of the following components: dextran polymers, cyclodextrin crosslinkers comprising matrix metalloprotease cleavable sites, collagen I, collagen III, collagen VI and fibronectin.

In certain embodiments, the stromal compartment permits penetration and invasion of an embryo in a physiological three-dimensional scaffold in vitro. In further embodiments, the outer luminal epithelial layer acts as site of anchorage to support initial embryo apposition and adhesion in vitro. In yet further embodiments, the functional secretory glandular endometrium provides histotrophic nourishment to support embryo growth during implantation in vitro. Overall, the current technology permits implantation of the human embryo in vitro and further development beyond this stage.

According to a further aspect of the invention, there is provided a three-dimensional endometrium assembloid obtainable by the methods defined herein.

According to a further aspect, there is provided an in vitro method of determining embryo implantation ability and/or window of receptibility to human embryo implantation, said method comprising the steps of:
(i) providing the three-dimensional endometrium assembloid defined herein, wherein the endometrial stromal cells and endometrial epithelial cells have been isolated from an endometrial biopsy obtained from a subject;
(ii) stimulating the endometrium assembloid using one or more of oestrogen, progesterone and cyclic AMP (CAMP), thereby rendering the endometrium assembloid receptive to embryo implantation; and
(iii) culturing a human stem cell based embryo-like (blastoid) or human blastocyst donated by an IVF patient in the presence of the stimulated endometrium assembloid, thereby allowing the blastoid or blastocyst to implant into the endometrium assembloid in vitro,
optionally wherein the blastocyst is isolated from the subject or the blastoid is derived from cells of the subject.

In a yet further aspect of the invention, there is provided a method of screening for an embryo implantation-modulating agent, said method comprising the steps of:
(i) providing the three-dimensional endometrium assembloid defined herein;
(ii) stimulating the endometrium assembloid using one or more of oestrogen, progesterone and cyclic AMP (CAMP) in the presence and/or absence of a test agent; and
(iii) culturing a human stem cell based blastoid or human blastocyst donated by an IVF patient in the presence of the stimulated endometrium assembloid,
wherein a difference between the implantation of the blastoid or blastocyst into the endometrium assembloid stimulated in the presence of the test agent and the implantation of the blastoid or blastocyst into the endometrium assembloid stimulated in the absence of the test agent is indicative of the implantation-modulating effect of said agent.

According to another aspect of the invention, there is provided the use of three sequential human embryo media (HEM) for culturing human stem cell based embryo-like (blastoids) or human blastocysts, comprising:
(i) culturing human stem cell based embryo-like (blastoids) or human blastocysts for between 5 and 9 days post-fertilisation or equivalent in human embryo medium 1 (HEM-1);
(ii) culturing the human blastoids or human blastocysts from step (i) for between 9 and 12 days post-fertilisation or equivalent in human embryo medium 2 (HEM-2); and
(iii) culturing the human blastoids or human blastocysts from step (ii) beyond 12 days post-fertilisation or equivalent in human embryo medium 3 (HEM-3).

### BRIEF DESCRIPTION OF THE FIGURES

FIG. 1—Construction of a three-dimensional endometrium assembloid. A) Human endometrial stromal cells and epithelial glandular were isolated from endometrial biopsies from healthy patients. Stromal cells were grown in culture in 2D culture flasks. Epithelial cells were embedded in Geltrex and propagated as glandular organoids. B-C) Method of generating an in vitro endometrium assembloid, comprising the steps of: (i) growing endometrial stromal cells inside the hydrogel; and (ii) layering cells from endometrial epithelial organoids on top of the cultured endometrial stromal cells in hydrogel. D) Stromal cells grow in 3D inside the hydrogel matrix, forming a network of interconnected cells, constituting the stromal compartment. E) Epithelial cells from endometrial organoids generate a continuous epithelial monolayer covering the entire surface of the stromal compartment. F) Cells in the luminal epithelial layer are enriched for acetylated alpha-tubulin. G) Formation of epithelial pockets throughout the luminal epithelial layer, resembling the glandular invaginations of the endometrium. H) Top view of the co-culture with a surface epithelial layer enriched for E-Cadherin on top hydrogel matrix containing cells positive for Vimentin. I) Schematic and cross view of the three-dimensional endometrium assembloid comprising a stromal compartment, an outer luminal epithelial layer and a glandular-like endometrium.

FIG. 2—Endometrial assembloid is responsive to hormonal stimulation and acquires features of tissue receptivity. A) Endometrial stromal cells in 3D display fibroblastic elongated appearance in proliferative phase (E2 stimulation, 4 days) while rounded epithelioid morphology in secretory phase (E2, P4 and CAMP stimulation, 8 days). B) Morphometric analysis of length/width ratio: significant cell shape change from elongated (median=9.14) to cuboidal (median=4.44), Mann Whitney Test **** p<0.0001, (number of cells, n=129 (proliferative), n=199 (secretory), 6 independent experiments). C) Quantification by ELISA of levels of prolactin (PRL) secreted into the culture medium (measured in the top well) before hormonal stimulation (unstimulated), in proliferative phase (E2 stimulation, 4 days) and secretory phase (E2, P4 and CAMP stimulation, at 4 and 8 days). Significant increase in PRL secretion at 8 days of secretory phase compared to unstimulated control (*p=0.0134) and proliferative phase (*p=0.0247): Kruskal-Wallis test corrected by Dunn's (11 independent experiments). D) Quantification of the levels of insulin-like growth factor binding protein 1 (IGFBP1): significant increase in IGFBP1 secretion at 8 days of secretory phase compared to unstimulated (****p<0.0001) and proliferative phase (***p=0.0004), and at 4 days of secretory phase compared to unstimulated (**p=0.0057): Kruskal-Wallis test corrected by Dunn's (10 independent experiments). E) Reconstruction in 3D of the surface epithelium. F) Morphometric analysis of epithelial endometrial cells: increase in surface area from proliferative (median=75.67 μm²) to secretory phase (median=147.13 μm²), Mann Whitney Test **** p<0.0001, (number of cells, n=2788 (proliferative), n=1597 (secretory), 6 independent experiments). G) Upon hormonal stimulation a subpopulation of epithelial cells lining the invagination becomes secretory, producing glycodelin (PAEP). H) Quantification by ELISA of PAEP, secreted in the top culture medium. Significant increase in PAEP levels at 8 days of secretory phase compared to unstimulated control (***p=0.0002) and to proliferative phase (*p=0.0436), and at 4 days of secretory phase compared to unstimulated (**p=0.008): Kruskal-Wallis test corrected by Dunn's (10 independent experiments). I) Cross-section view shows the appearance of pinopodes following hormonal stimulation. J) Presence of mono- and multi-ciliated cells within the epithelium. Scale bars: 100 μm (A), 50 μm (B, G), 25 μm (G (zoom), H and I), 10 μm (I). Images labelled (z-proj) display max projection of the z-stack; z-section represents single plane; 3D represents reconstruction.

FIG. 3—Implantation of human embryos in vitro. A) Schematic of the experimental set up: human blastocysts at 5 d.p.f were transferred into the endometrial assembloid, undergo adhesion and invasion inside the hydrogel and allowing development to post-implantation stage 14 d.p.f. B) Supernumerary human blastocysts at 5 d.p.f were thawed, transferred into the endometrial assembloid and allowed to hatch naturally from zona pellucida, which occurred within 24 h from the transfer (see picture at 6 d.p.f). By 7 d.p.f the blastocoel cavity undergoes significant expansion and that blastocyst orients upside-down facing the luminal endometrium. This is followed by firm attachment to the endometrium at ˜8 d.p.f as shown by the collapse of the blastocoel cavity and formation of an implantation site. C) Adhesion to the endometrial surface occurred at 8 d.p.f in the majority of cases (46%), despite significant variabilities exist in the time of adhesion. D) Steady increase in human chorionic gonadotropin (hCG) secretion by the embryo into the culture medium over time. E) Implanted human embryo at 10 d.p.f.: formation of rosette configuration at the centre of the epiblast (OCT4+), presence of hypoblast layer as shown by the columnar and highly packed morphology of the SOX17+ cells lining the basal domain of the epiblast, and formation of a yolk sac cavity demarcated by flat and dispersed SOX17+ cells. F) Human embryo at 14 d.p.f.: expansion of the trophectoderm plug, appearance of the pro-amniotic cavity, expansion of the yolk sac cavity (SOX17+ cells). G) Major structural transformation of the trophoblast at 14 d.p.f: appearance of large plate-like assemblies of multinucleated cells and appearance of population of KRT7 expressing cells giving rise to intricate branches radially from the implantation centre. Zoom in view of the primary connections between embryo and maternal stromal cells and active remodelling of tissues.

FIG. 4—Assembly of endometrium assembloid in three-dimensions. A) Selection of major extracellular matrix components to incorporate into the hydrogel matrix by analysis of gene expression profile of endometrial biopsies. B) Culture of endometrial organoids inside Geltrex. C) Fragmentation of endometrial organoids and layering of organoids fragments forming epithelial outgrowth. D) Cross section of the endometrial assembloid showing formation of a stromal compartment beneath surface epithelial layer.

FIG. 5—Morphometric analysis of stromal cells and secretions. A) Morphology of endometrial stromal cells at secretory phase. B) Morphometric analysis: quantification of cell length and width. C) Length/width ratio values in each independent experiment: proliferative 1 (number of cells, n=50), proliferative2 (n=40), proliferative3 (n=39), secretory1 (n=66), secretory2 (n=78) and secretory3 (n=55). D) Comparison of cell length in proliferative (median length=105.4 μm) vs secretory (median length=83.39 μm): Mann Whitney Test * p=0.0128, (number of cells, n=129 (proliferative), n=199 (secretory). E) Comparison of cell width in proliferative (median width=11.39 μm) vs secretory (median length=19.97 μm): Mann Whitney Test **** p<0.0001. Refer to FIG. 2b. F) Quantification by ELISA of levels of prolactin (PRL) secreted measured in bottom medium: before hormonal stimulation (unstimulated), in proliferative phase (E2 stimulation, 4 days) and secretory phase (E2, P4 and CAMP stimulation, at 4 and 8 days). Significant increase in PRL secretion at 4 days of secretory phase compared to unstimulated control (*p=0.0207), at 8 days of secretory phase compared to unstimulated (***p=0.0002) and to proliferative phase (*p=0.0326): Kruskal-Wallis test corrected by Dunn's (11 independent experiments). G) Quantification of levels of PRL secreted in top and bottom media. H) Quantification levels of insulin-like growth factor binding protein 1 (IGFBP1) secreted measured in bottom medium. Significant increase in IGFBP1 secretion at 4 days of secretory phase compared to unstimulated control (*p=0.0407), at 8 days of secretory phase compared to unstimulated (****p<0.0001) and to proliferative phase (**p=0.0023): Kruskal-Wallis test corrected by Dunn's (10 independent experiments). I) Quantification of levels of IGFBP1 secreted in top and bottom media. Scale bars: 50 μm (A).

FIG. 6—Morphometric analysis of epithelial cells. A-B) Morphometric analysis of cells of the surface epithelium was performed by manual segmentation. Refer to FIG. 2E. C) Quantification of cell surface area in each independent experiment: proliferative1 (number of cells, n=859), proliferative2 (n=1572), proliferative3 (n=357), secretory1 (n=1185), secretory2 (n=267) and secretory3 (n=145). D) Quantification of cell perimeters in proliferative (median=34.61 μm) vs secretory (median=49.74 μm): Mann Whitney Test **** p<0.0001, (number of cells, n=2788 (proliferative), n=1597 (secretory), 6 independent experiments). E) Quantification of cell roundness (4*area/(π*major_axis\hat{ }2) in proliferative (median=0.6315) vs secretory (median=0.5980): Mann Whitney Test **** p<0.0001, (number of cells, n=2788 (proliferative), n=1597 (secretory), 6 independent experiments). Roundness gives a value directly correlated to the aspect ratio. Roundness ˜0 indicates elongated morphology, roundness ˜1 indicates perfect circle. F) Quantification of aspect ratio (AR) (major axis/minor axis of the best fit ellipse) in proliferative (median=1.584) vs secretory (median=1.673): Mann Whitney Test **** p<0.0001, (number of cells, n=2788 (proliferative), n=1597 (secretory), 6 independent experiments). AR=1 corresponds to perfect circle. G) Quantification of circularity (4π*area/perimeter\hat{ }2) in proliferative (median=0.7920) vs secretory (median=0.7520): Mann Whitney Test **** p<0.0001, (number of cells, n=2788 (proliferative), n=1597 (secretory), 6 independent experiments). Circularity measures shape roughness and depends directly on perimeter: cell round and smooth (circularity ˜1) vs cell with rough perimeter and irregular borders (circularity ˜0) H) Quantification of solidity (area/convex area) in proliferative (median=0.9820) vs secretory (median=0.9550): Mann Whitney Test **** p<0.0001, (number of cells, n=2788 (proliferative), n=1597 (secretory), 6 independent experiments). Solidity describes the extent to which a cell is convex (solidity ˜1) or concave (solidity ˜0) based on the area enclosed by a convex hull. Scale bar: 50 μm (A).

FIG. 7—Epithelial secretions and endometrial receptivity. A) Immunofluorescence analysis of the epithelium before hormonal stimulation compared to the secretory phase (E2, P4 and cAMP, 8 days), showing a subset of epithelial secretory cells producing glycodelin. B) Appearance of cilia on the surface of the endometrial epithelium after hormonal stimulation (E2, P4 and cAMP, 8 days): a subset of cells displays a single cilium, a subset of cells displays multiple cilia, the majority of cells do not display cilia. Scale bars: 50 μm (A), 10 μm (B).

FIG. 8—Implantation of human embryos in vitro (extended). A) Quantification over time (24 h, 48 h and 72 h culture) of percentages of blastoids unattached/floating, apposed to the endometrial surface, and implanted inside the endometrial scaffold. (7 replicates, total number of blastoids n=66). I) Example of implanted blastoids at 72 hours of culture. The site of implantation can be visualised by GFP+. The epiblast is marked by OCT4+. C) Quantification of the area of the implantation site in human embryos over time, displaying mean values±SEM (standard error of the mean). The area quantification includes only implanted embryos. D) Quantification of the implantation site in blastoids after 72 hours of culture. Box represents the 25th and 75th percentiles intervals, middle line is the median, and whiskers show the minimum and maximum values. The median area of implanted blastoids is comparable to the size of implantation area in human embryos between 8-9 d.p.f. although there is substantial variation. Scale bars: 50 μm and 100 μm (B). Images labelled (z-proj) display max projection of the z-stack; z-section represents single plane; 3D represents reconstruction.

FIG. 9—Implanted human embryos at 14 d.p.f. A) Top view of the implantation site at 14 d.p.f: the embryo is marked by epiblast (OCT4+), hypoblast (SOX17+) and trophoblast (GATA3+, KRT7+), the surrounding decidualised endometrial stromal cells are marked by VIM. Right inserts: sites of contact between embryo and maternal endometrial cells. Primary syncytium as shown by the large plate-like assemblies of multinucleated cells around the entire trophoblast plate. Lacunae can be visualised. KRT7 expressing cells giving rise to intricate trophoblast strands branching radially and laterally. B) Presence of multi-nucleated cells positive for SDC1+ confirming syncytial transformation of some of the trophoblast cells. C) Zoom in views of the primary connections between embryo (KRT+) and maternal stromal cells (VIM+). Focal anchorage and active remodelling of tissues can be visualized at the sites of contact between embryo and stromal cells. D) Presence and organisation of HLA-G+ extravillous trophoblast cells at the implantation site. E) The secretion of glycodelin increases over time following implantation of human embryos, indicating a potential cross-communication between the implanted embryo and endometrium that results in increased endometrial secretions. Box represents the 25th and 75th percentiles intervals, middle line is the median, and whiskers show the minimum and maximum values. Scale bars: 100 μm (B-D).

### DETAILED DESCRIPTION OF THE INVENTION

The present invention is based on the development of a culture method to generate a new in vitro engineered endometrial scaffold, herein referred to as an “endometrium assembloid”. This multi-cellular three-dimensional uterus appears to closely recapitulate the complex cytoarchitectures of the human endometrium and to adopt features of tissue receptivity reminiscent of the window of implantation. Supernumerary human blastocysts and stem-cell embryo models (e.g. blastoids) are able to successfully implant into the receptive endometrium assembloid and undergo physiological development to equivalent stages as the specimens from the Carnegie Institute. To this end, the endometrium assembloid comprises a stromal compartment that permits penetration and invasion of an embryo in a physiological three-dimensional scaffold in vitro, an outer luminal epithelial layer which acts as site of anchorage to support initial embryo apposition and adhesion in vitro, and a functional secretory glandular endometrium which provides histotrophic nourishment to support embryo growth during implantation in vitro.

Thus according to a first aspect of the invention, there is provided a method of generating an in vitro endometrium assembloid, said method comprising the steps of:
(i) growing endometrial stromal cells inside a hydrogel matrix; and
(ii) layering cells and/or fragments from endometrial epithelial organoids on top of the cultured endometrial stromal cells in hydrogel matrix of step (i);
thereby generating a three-dimensional endometrium assembloid comprising a stromal compartment, an outer luminal epithelial layer and a functional secretory glandular endometrium,
wherein the hydrogel matrix comprises any one or more of the following components: dextran polymers, cyclodextrin crosslinkers comprising matrix metalloprotease cleavable sites, collagen I, collagen III, collagen VI and fibronectin.

References herein to “assembloid” refer to a three-dimensional tissue structure that has been created in vitro and recapitulates at least the structural features of the naturally occurring tissue on which it is based or from which the cells used to generate the assembloid have been obtained. In addition to the structure of uterine tissue in vivo, the endometrium assembloid defined herein further recapitulates the function of said tissue/endometrium, providing a highly useful tool for studying and/or manipulating embryo implantation in vitro. Thus in certain embodiments, the endometrium assembloid provided herein recapitulates both the structural and functional features of in vivo endometrium, in particular human endometrium. In further particular embodiments, the methods described herein generate a human in vitro endometrium assembloid. It will be readily appreciated that “assembloid” herein may also be referred to as a culture system or model, or as an in vitro system or model. Therefore, the endometrium assembloid may also be referred to as an endometrium culture system/model or as an in vitro endometrium system/model.

In one embodiment, the method comprises the step of: (i) growing endometrial stromal cells inside a hydrogel matrix in 3D.

It will be appreciated that the hydrogel matrix comprises any one or more of the following components: dextran polymers, cyclodextrin (CD) crosslinkers comprising matrix metalloprotease cleavable sites, and extracellular matrix molecules such as collagen I, collagen III, collagen VI and fibronectin. In one embodiment, the hydrogel matrix comprises two or more of the following components: dextran polymers, cyclodextrin crosslinkers comprising matrix metalloprotease cleavable sites, collagen I, collagen III, collagen VI and fibronectin. In one embodiment, the hydrogel matrix comprises three or more of the following components: dextran polymers, cyclodextrin crosslinkers comprising matrix metalloprotease cleavable sites, collagen I, collagen III, collagen VI and fibronectin. In one embodiment, the hydrogel matrix comprises four or more of the following components: dextran polymers, cyclodextrin crosslinkers comprising matrix metalloprotease cleavable sites, collagen I, collagen III, collagen VI and fibronectin. In one embodiment, the hydrogel matrix comprises five or more of the following components: dextran polymers, cyclodextrin crosslinkers comprising matrix metalloprotease cleavable sites, collagen I, collagen III, collagen VI and fibronectin. In one embodiment, the hydrogel matrix comprises each of the following components: dextran polymers, cyclodextrin crosslinkers comprising matrix metalloprotease cleavable sites, collagen I, collagen III, collagen VI and fibronectin.

The hydrogel matrix provides a three-dimensional structure/backbone in which the endometrial stromal cells are cultured, and comprises dextran polymers, cyclodextrin crosslinkers comprising matrix metalloprotease cleavable sites, collagen I, collagen III, collagen VI and fibronectin. The presence of dextran polymers and cyclodextrin crosslinkers enables modulation of the elastic modulus of the hydrogel matrix to a predefined stiffness (i.e. any specific stiffness, rigidity or tension). An elastic modulus is the unit of measurement of a substance's resistance to being deformed elastically (i.e. non-permanently) when a stress is applied. The higher the elastic modulus, the stiffer the material. In one embodiment, the hydrogel matrix comprises an elastic modulus of at least 200 Pa, such as about 250 Pa.

The elastic modulus of about 250 Pa defined herein recapitulates the tissue stiffness of the natural endometrium, as measured during the mid-secretory phase of the menstrual cycle (Abbas et al. (2019) Hum. Reprod., 34(10):1999-2008, doi: https://doi.org/10.1093/humrep/dez139). The dextran polymers also display slow gelation properties, allowing the incorporation of endometrial stromal cells. Matrix metalloprotease cleavable sites in the crosslinker provide tissue degradability. Such degradability allows the penetration and invasion by trophoblast cells during implantation, ensuring the burrowing of the embryo into the stromal layer through active tissue degradation. Collagen I, collagen III, collagen VI and fibronectin are comprised in the hydrogel matrix at relative ratios to closely mimic the biochemical composition of the extracellular matrix of the natural endometrium (Aplin, Charlton & Ayad (1988) Cell Tissue Res., 253(1):231-240, doi: https://doi.org/10.1007/bf00221758).

For example, suitable non-limiting ratios include 45% collagen III, 35% collagen I, 10% collagen VI and 10% fibronectin. In one specific embodiment, the hydrogel is a commercially available composition, such as TrueGel3D Hydrogel Kit (Catalogue Number TRUE7).

This closer mimicking of the natural endometrium extracellular matrix contrasts to previous models which are based on less well-defined extracellular matrix extracts such as Matrigel (Arnold et al. (2002) Cancer Res., 62(1):79-88, PMID: 11782363; and Buck et al. (2015) Hum. Reprod., 30(4):906-916, doi: https://doi.org/10.1093/humrep/dev011), or single defined components such as collagen (Bentin-Ley et al. (1994) Reproduction, 101(2):327-332, doi: https://doi.org/10.1530/jrf.0.1010327) or fibrin (Wang et al. (2012) Mol. Hum. Reprod., 18(1):33-43, doi: https://doi.org/10.1093/molehr/gar064).

Thus in a particular embodiment, the hydrogel matrix comprises dextran polymers, cyclodextrin crosslinkers comprising matrix metalloprotease cleavable sites, collagen I, collagen III, collagen VI and fibronectin, and comprises an elastic modulus of about 250 Pa.

In one embodiment, the endometrial stromal cells are isolated from endometrial biopsies (see methods and Michalski et al. (2018) Journal of Visualized Experiments, 139: e57684). Thus in certain embodiments, the endometrial stromal cells are primary endometrial stromal cells. Methods for isolating endometrial stromal cells from endometrial biopsies will be well known to the person skilled in the art and may include, without limitation, the use of labelled antibodies which bind to cell-specific markers or a cell-specific combination of markers (in particular cell surface markers) for magnetic-activated cell sorting (MACS) and/or fluorescence-activated cell sorting (FACS). Such cell-specific markers for isolating endometrial stromal cells are stromal cell-specific surface markers. In a further embodiment, the endometrial biopsies and/or the primary endometrial stromal cells are from a mammalian subject, in particular a human subject. Thus in particular embodiments, the endometrial stromal cells, such as the primary endometrial stromal cells, are human endometrial stromal cells. In other embodiments, the endometrial stromal cells are from a cell line, such as an immortalised human endometrial stromal cell line (e.g. T0553, SHT290, CRL-4003, KC02-44D and those described in Krikun et al. (2004) Endocrinology, 145(5):2291-2296, doi: https://doi.org/10.1210/en.2003-1606). Such immortalised human endometrial stromal cell lines (e.g. T0553 in particular) are unique compared to other human cell lines in that they 1) display normal karyotype and 2) respond to hormone stimulation and retain the morphological pattern and biochemical endpoints of decidualization after stimulation with hormones, such as oestradiol and medroxyprogesterone.

In a further embodiment, step (i) comprises growing the endometrial stromal cells inside three-dimensions, such as in a transwell system. In yet further embodiments, step (i) comprises growing the endometrial stromal cells inside the hydrogel matrix, such as in three-dimensions, for about 4 days or for at least 4 days, such as between 4 and 20 days. In still further embodiments, step (i) comprises growing the endometrial stromal cells inside the hydrogen matrix, such as in three-dimensions, for about 5 days, about 6 days or for at least 5 days, such as between 5 and 20 days or between 5 and 6 days. In other embodiments, the endometrial stromal cells are cultured in step (i) for a period of time until morphological properties are observed which are characteristic of endometrial stromal cells in natural endometrium and/or the natural stromal layer of natural endometrium. Such morphological properties include extensive elongation and generation of a highly ordered interconnected cellular network throughout the depth of the hydrogel matrix.

In one embodiment, the method comprises the step of: (ii) layering cells and/or fragments from endometrial epithelial organoids on top of the cultured endometrial stromal cells in hydrogel matrix of step (i). Such layering of endometrial epithelial cells and/or fragments on top of the cultured endometrial stromal cells recapitulates the structured epithelial layer of natural endometrium by formation of the epithelium outgrowth, and the use of cells and/or fragments from endometrial endothelial organoids induces tissue assembly by sequential layering deposition. A continuous monolayer covering the endometrial stromal cells in hydrogel matrix is thus formed. After layering, the endometrial epithelial cells may be cultured for at least 1 day, such as between 1 and 12 days. Thus in a further embodiment, the method additionally comprises the step of: (iii) growing the endometrial stromal cells inside hydrogel matrix and layered cells and/or fragments from endometrial epithelial organoids for at least 1 day, such as between 1 and 12 days. In a further embodiment, growing step (iii) is for at least 3 days. In a yet further embodiment, growing step (iii) is for at least 4 days. In another embodiment, growing step (iii) is for between 3 and 4 days. In a yet other embodiment, growing step (iii) is for a period of time until epithelial outgrowths are observed originating from the organoid fragments and/or a single continuous epithelial monolayer is formed that covers the endometrial stromal cells in hydrogel matrix (i.e. covers the stromal compartment).

In one embodiment, the endometrial epithelial cells are from endometrial biopsies. Thus in certain embodiments, the endometrial epithelial cells are primary endometrial epithelial cells. Methods for isolating endometrial epithelial cells from endometrial biopsies will be well known to the person skilled in the art and may include, without limitation, the use of labelled antibodies which bind to cell-specific markers or a cell-specific combination of markers (in particular cell surface markers) for magnetic-activated cell sorting (MACS) and/or fluorescence-activated cell sorting (FACS). Such cell-specific markers for isolating endometrial epithelial cells are epithelial cell-specific surface markers. In a further embodiment, the endometrial biopsies and/or the primary endometrial epithelial cells are from a mammalian subject, in particular a human subject. Thus in particular embodiments, the endometrial epithelial cells, such as the primary endometrial epithelial cells, are human endometrial epithelial cells. In other embodiments, the endometrial epithelial cells are from a cell line, such as an immortalised human endometrial epithelial cell line (e.g. hEM3, EnCa101AE and ECC1).

In particular embodiments, the cells and/or fragments from endometrial epithelial cell organoids are from an epithelial organoid culture system. Said organoid culture system may be that described in Turco et al. (2017) Nat. Cell Biol., 19(5):568-577 (doi: https://doi.org/10.1038/ncb3516).

Thus in one embodiment, the organoid culture system comprises the step of: (i) resuspending tissue isolates enriched for epithelial cells in a solubilised basement membrane preparation comprising laminin, collagen IV, heparin sulphate proteoglycans, entactin/nidogen and growth factors. In certain embodiments, the tissue isolates enriched for epithelial cells are glandular elements obtained from endometrial and/or decidual tissue. Thus, resuspending tissue isolates enriched for epithelial cells may comprise resuspending glandular elements obtained from endometrial and/or decidual tissue (e.g. biopsies) in a solubilised basement membrane preparation comprising laminin, collagen IV, heparin sulphate proteoglycans, entactin/nidogen and growth factors. In a further embodiment, the tissue isolates enriched for epithelial cells are obtained from endometrial and/or decidual tissue biopsies. Obtaining tissue isolates enriched for epithelial cells, such as glandular elements, from endometrial and/or decidual tissue biopsies may be performed by enzymatic digestion. Such digestion disaggregates stromal cells from the epithelial cell-containing elements of the biopsy as the stromal cells disaggregate more readily in the presence of enzyme, such as collagenase and/or dispase, than epithelial cell-containing elements (e.g. glandular elements). Thus in one embodiment, the enzymatic digestion is by collagenase and/or dispase, such as a solution containing both collagenase and dispase. Enzymatic digestion is therefore allowed to proceed for a period of time until stromal cells have disaggregated but prior to any significant disaggregation of epithelial cell-containing elements. Thus in a further embodiment, enzymatic digestion is halted prior to disaggregation of epithelial cell-containing elements of the biopsy, such as prior to disaggregation of glandular elements. The halting of enzymatic digestion may be by addition of an excess of diluent (e.g. phosphate buffered saline (PBS) or a cell culture medium) or by washing. Thus in a particular embodiment, the tissue isolates enriched for epithelial cells are obtained from endometrial and/or decidual tissue biopsies by enzymatic digestion to disaggregate stromal cells, wherein said enzymatic digestion is halted prior to disaggregation of epithelial cell-containing elements of the biopsy, such as prior to disaggregation of glandular elements.

In one embodiment, the solubilised basement membrane preparation comprises laminin, collagen IV, heparin sulphate proteoglycans, entactin/nidogen and growth factors. Use of a solubilised basement membrane preparation allows the epithelial cell-enriched tissue isolates to be cultured in three-dimensions, such as in droplets. In a further embodiment, the solubilised basement membrane preparation is extracted from Engelbreth-Holm-Swarm (EHS) mouse sarcoma. An example of such solubilised basement membrane preparations is Matrigel, which is obtainable commercially from Corning® and other suppliers.

In a further embodiment, the organoid culture system comprises the step of: (ii) culturing the resuspended tissue isolates of step (i) in a culture medium comprising epidermal growth factor (EGF), noggin, R-spondin-1, nicotinamide, hepatocyte growth factor (HGF), the Alk3/4/5 inhibitor A83-01 and optionally fibroblast growth factor 10 (FGF10). Nicotinamide and the Alk3/4/5 inhibitor, A83-01, that blocks the TGFβ pathway have been previously reported as crucial in the establishment and/or long-term culture of other human organoid systems (Sato et al. (2011) Gastroenterology, 141(5):1762-1772, doi: https://doi.org/10.1053/j.gastro.2011.07.050; Karthaus et al. (2014) Cell, 159(1):163-175, doi: https://doi.org/10.1016/j.cell.2014.08.017; and Bartfeld et al. (2015) Gastroenterology, 148(1):126-136.e126, doi: https://doi.org/10.1053/j.gastro.2014.09.042). The combination of EGF, noggin, R-spondin-1, nicotinamide, HGF, A83-01 and FGF10 in the culture medium has been shown to give good expansion of cells from the isolated tissue isolates enriched for epithelial cells and a high yield of expanded organoids (Turco et al. (2017)).

Thus in a certain embodiment, the cells and/or fragments of endometrial epithelial organoids are from an epithelial organoid culture system comprising:
(i) resuspending tissue isolates enriched for epithelial cells, such as glandular elements obtained from endometrial and/or decidual tissue, in a solubilised basement membrane preparation comprising laminin, collagen IV, heparin sulphate proteoglycans, entactin/nidogen and growth factors; and
(ii) culturing the resuspended tissue isolates of step (i) in a culture medium comprising epidermal growth factor (EGF), noggin, R-spondin-1, nicotinamide, hepatocyte growth factor (HGF), the Alk3/4/5 inhibitor A83-01 and optionally fibroblast growth factor 10 (FGF10).

In one embodiment, step (ii) comprises culturing the resuspended tissue isolates for at least 7 days. In a particular embodiment, step (ii) comprises culturing for 7 days. In a further embodiment, step (ii) comprises culturing for up to and including 14 days, such as for 14 days. In a yet further embodiment, step (ii) comprises culturing for between 7 and 14 days. In other embodiments, the resuspended tissue isolates are cultured in step (ii) for a period of time until properties (e.g. morphological properties) are observed which are characteristic of endometrial epithelial cells in natural endometrium and/or the natural epithelial layer of natural endometrium. Such properties include the bi-potential capability to generate luminal and glandular lineages, and the self-organisation into a superficial luminal epithelium continuous to glandular-like invaginations. Thus, the cells and/or fragments from endometrial epithelial culture organoids obtained according to the epithelial organoid culture system described herein retain the bi-potential capability to generate luminal and glandular lineages, and are able to self-organise into a superficial luminal epithelium continuous to glandular-like invaginations in culture, i.e. are similar to the natural epithelial layer of natural endometrium. Such cells and/or fragments and the endometrial epithelial culture organoids from which they are derived are therefore highly heterogeneous in shape and display numerous invaginations after layering on top of the cultured endometrial stromal cells in hydrogel matrix, anatomically resembling the deep-reaching uterine glands seen in natural endometrium. Thus, the cells and/or fragments and the endometrial epithelial culture organoids from which they are derived are capable of producing a continuous epithelial surface layer, which is continuous from the outer layer into the invaginated glands. In one embodiment, the outer luminal epithelial layer generated from the endometrial epithelial culture organoid cells and/or fragments comprises acetylated α-tubulin rich cells.

Thus according to the first aspect of the invention, a three-dimensional endometrium assembloid comprising a stromal compartment, an outer luminal epithelial layer and a functional secretory glandular endometrium is thereby generated. As described hereinbefore, the stromal compartment is generated by the culturing of endometrial stromal cells in a hydrogel matrix as in step (i) of the first aspect of the present invention. Such stromal compartment is therefore three-dimensional and permits penetration and invasion of an embryo in this physiological three-dimensional scaffold in vitro. The outer luminal epithelial layer and functional secretory glandular endometrium are both generated from the layering of cells and/or fragments from endometrial epithelial organoids on top of the cultured endometrial stromal cells in hydrogel matrix (i.e. on top of the stromal cell compartment) as in step (ii) of the first aspect of the invention. The outer luminal epithelial layer and functional secretory glandular endometrium are further generated by culturing the endometrial stromal cells in hydrogel matrix and layered cells and/or fragments from endometrial epithelial organoids (i.e. by additional step (iii) described herein). The outer luminal epithelial layer acts as site of anchorage to support initial embryo apposition and adhesion in vitro, and the functional secretory glandular endometrium provides histotrophic nourishment to support embryo growth during implantation in vitro. Thus in one embodiment, the outer luminal epithelial layer acts as site of anchorage to support initial embryo apposition and adhesion in vitro. In a further embodiment, the outer luminal epithelial layer comprises acetylated α-tubulin rich cells, such as cilia. In a yet further embodiment, the functional secretory glandular endometrium provides histotrophic nourishment to support embryo growth during implantation in vitro. In a still further embodiment, which may be preferred, the outer luminal epithelial layer acts as site of anchorage to support initial embryo apposition and adhesion and the functional secretory glandular endometrium provides histotrophic nourishment to support embryo growth during implantation in vitro.

As demonstrated herein, the endometrium assembloid generated by the methods of the invention recapitulates the functions of the natural uterine tissue/endometrium, in particular the human endometrium. Such functions include responsiveness to hormone stimulation. Thus in one embodiment, the endometrium assembloid undergoes proliferation in response to oestrogen. In other words, the endometrium assembloid is capable of recapitulating the proliferative phase of the menstrual cycle, driven by a peak in oestrogen levels. In a further embodiment, the endometrium assembloid undergoes decidualization of the stromal compartment and differentiation of the epithelial endometrial layer in response to oestrogen and progesterone stimulation in culture. In another embodiment, decidualization is in response to oestrogen, progesterone and cyclic AMP (CAMP). Thus, the endometrium assembloid is capable of differentiation towards the mid-secretory phase (mid-luteal phase) of the menstrual cycle, driven by oestrogen, progesterone and optionally CAMP, i.e. it is capable of decidualization and differentiation. During the differentiation from the proliferative phase to the mid-secretory phase (i.e. during decidualization), the endometrial stromal cells of the endometrium assembloid undergo extensive morphological transformations as shown by the shift from an elongated fibroblast-like morphology (during the proliferative phase in response to oestrogen stimulation) to a rounded epithelial-like morphology during the secretory phase (in response to oestrogen, progesterone and cAMP). During decidualization, the endometrial stromal cells of the endometrium assembloid further acquire a secretory-like phenotype, with increased secretion of prolactin and insulin-like growth factor binding protein 1 (IGFBP1). Both the morphological changes and acquisition of secretory capabilities in response to hormonal stimulation are central hallmarks reminiscent of the process of tissue decidualization of the natural human endometrium during the mid-secretory phase (Gellersen & Brosens (2014) Endocr. Rev., 35(6):851-905, doi: https://doi.org/10.1210/er.2014-1045). This is in contrast to decidualization of the endometrium in other animal models such as rodents which is induced by mechanical stimulations, such as the presence of the embryo/blastocyst (Ramathal et al. (2010) Semin. Reprod. Med., 28(1):17-026, doi: https://doi.org/10.1055/s-0029-1242989). Thus in one embodiment, decidualization comprises morphological transformation and reorganisation of the actin cytoskeleton in the cells of the stromal compartment, and/or secretion of prolactin and/or insulin-like growth factor binding protein 1 (IGFBP1) by the cells of the stromal compartment.

The endometrial epithelial cells of the endometrium assembloid also undergo morphological remodelling during the secretory phase/differentiation, with increased apical cell surface area and perimeter in response to hormonal stimulation. Also upon hormonal stimulation, the endometrial epithelial cells of the endometrium assembloid adopt a more elongated morphology, as shown by decreased roundness and increased aspect ratio, and display irregular borders due to decreased circularity and solidity compared to a smoother and more circular morphology during the proliferative phase. These morphological changes are consistent with the acquisition of a secretory phenotype, which is in turn shown by a progressive increase in glycodelin secretions by the endometrium assembloid. Glycodelin is a major constituent of the secretions produced by the endometrial glands in vivo which are known collectively as the “uterine milk” (Burton, Cindrova-Davies & Turco (2020) Placenta, 102:21-26, doi: https://doi.org/10.1016/j.placenta.2020.02.008; and Burton et al. (2002) J. Clin. Endocrinol. Metab., 87(6):2954-2959, doi: https://doi.org/10.1210/jcem.87.6.8563). In response to stimulation with oestrogen, progesterone and cAMP (i.e. during decidualization), the epithelial cells at the border of the invaginations observed in the endometrium assembloid are positive for glycodelin and are thus glandular-like in nature. Thus in a further embodiment, differentiation comprises morphological remodelling of the cells of the outer epithelial layer, and/or secretion of glycodelin and/or uterine milk by the cells of the outer luminal epithelial layer and functional secretory glandular endometrium.

It will be appreciated that due to the structure of the endometrium assembloid described herein, secretions are released into the culture medium and are therefore accessible to an embryo or blastoid/blastocyst in vitro. This is in contrast to previous models based on endometrial organoids in which secretions are trapped in the lumen due to their inverted basal-out apical-polarity (Buck et al. (2015) Hum. Reprod., 30(4):906-916, doi: https://doi.org/10.1093/humrep/dev011; Rawlings et al. (2021) Elife, 10:e69603, doi: https://doi.org/10.7554/elife.69603; and_Simintiras et al. (2021) Proc. Sci., 118(15):e2026804118, doi: Natl. Acad. https://doi.org/10.1073/pnas.2026804118). Thus in one embodiment, the secretions are accessible to the embryo in vitro. In a further embodiment, the secretions are accessible to the blastoid or blastocyst in vitro. Such direct access of the embryo or blastoid/blastocyst to these glandular secretions is an essential requisite which ensures histotrophic nourishment of the conceptus during most of the first human trimester, before the haemochorial placenta becomes fully functional (Burton, Cindrova-Davies & Turco (2020); and Burton et al. (2002)).

Another feature of the endometrial epithelial cells of the endometrium assembloid is the formation of pinopodes on the apical surface during differentiation. Pinopodes are bleb-like structures which are a major hallmark of endometrial receptivity, and their appearance is limited to the brief receptive window of implantation (Quinn et al. (2020) Mol. Cell. Endocrinol., 501:110644, doi: https://doi.org/10.1016/j.mce.2019.110644; and Bentin-Ley et al. (1999) Hum. Reprod., 14(2):515-520, doi: https://doi.org/10.1093/humrep/14.2.515). They also appear to be directly involved in the adhesion of the blastocyst to the endometrial surface in vivo, and have been proposed to act as primary sites of anchorage for embryo apposition and adhesion (Bentin-Ley et al. (1999); and Stavreus-Evers et al. (2002) Mol. Hum. Reprod., 8(8):765-769, doi: https://doi.org/10.1093/molehr/8.8.765). As demonstrated herein, during differentiation of the endometrium assembloid pinopodes are formed on the apical surface of the outer luminal epithelial layer. Thus in one embodiment, differentiation comprises formation of pinopodes on the apical surface of the outer luminal epithelial layer.

In a further embodiment, the outer endometrial epithelial cells comprise ciliated cells. Such ciliated cells may comprise single cilium or multiple cilia, and the presence of cilia may be identified by acetylated α-tubulin rich cells as described hereinbefore. In vivo, ciliated cells are present across the entire menstrual cycle (More & Masterton (1976) Reproduction, 47(1):19-24, doi: https://doi.org/10.1530/jrf.0.0470019; and Masterton, Armstrong & More (1975) J. Reprod. Fertil., 42(3):537-540, doi: https://doi.org/10.1530/jrf.0.0420537) both within the luminal epithelium and on glandular-like invaginations, interspersed among the more abundant population of neighbouring unciliated epithelial cells. Thus in one embodiment, the outer luminal epithelial layer and functional secretory glandular endometrium comprise ciliated cells, such as cells comprising a single cilium and/or multiple cilia.

It will be readily understood that the methods described hereinbefore for generating an endometrium assembloid are performed in vitro, i.e. in culture (e.g. mammalian cell culture) and not in/on the human or animal body. Methods on stromal and epithelial cells isolated from an endometrial biopsy can be considered to be performed ex vivo.

According to a further aspect of the invention, there is provided a three-dimensional endometrium assembloid obtainable by the methods defined herein. Thus according to one aspect, there is provided a three-dimensional endometrium assembloid obtained by the methods defined herein.

According to a yet further aspect of the invention, there is provided the use of three sequential human embryo media (HEM) for culturing human stem cell based embryo-like (blastoids) or human blastocysts, comprising:
(i) culturing human stem cell based embryo-like (blastoids) or human blastocysts for between 5 and 9 days post-fertilisation or equivalent in human embryo medium 1 (HEM-1);
(ii) culturing the human blastoids or human blastocysts from step (i) for between 9 and 12 days post-fertilisation or equivalent in human embryo medium 2 (HEM-2); and
(iii) culturing the human blastoids or human blastocysts from step (ii) beyond 12 days post-fertilisation or equivalent in human embryo medium 3 (HEM-3).

In one embodiment, HEM-1 comprises one or more of the following components: a basal medium (Advanced DMEM/F12), B27 supplement, N2 supplement, L-glutamine, antibiotic, N-Acetyl-L-cysteine (NAC), nicotinamide, charcoal stripped FBS, human serum albumin (HSA), hyaluronan, oestrogen (E2), progesterone (P4), insulin-like growth factor 1 (IGF-1), epidermal growth factor (EGF), human prolactin, human Chorionic Gonadotropin (hCG) and human Placental Lactogen (hPL). In a further embodiment, HEM-2 comprises one or more component of HEM-1 in addition to a ROCK inhibitor, glucose, Insulin-Transferrin-Selenium-Ethanolamine (ITS-X), non-essential amino acids, essential amino acids and an increased concentration of charcoal stripped FBS relative to HEM-1. In a yet further embodiment, HEM-2 comprises HEM-1 in addition to a ROCK inhibitor, glucose, Insulin-Transferrin-Selenium-Ethanolamine (ITS-X), non-essential amino acids, essential amino acids and an increased concentration of charcoal stripped FBS relative to HEM-1. In an alternative embodiment, HEM-2 comprises one or more component of HEM-1 in addition to glucose, Insulin-Transferrin-Selenium-Ethanolamine (ITS-X), non-essential amino acids, essential amino acids and an increased concentration of charcoal stripped FBS relative to HEM-1. In a further alternative embodiment, HEM-2 comprises HEM-1 in addition to glucose, Insulin-Transferrin-Selenium-Ethanolamine (ITS-X), non-essential amino acids, essential amino acids and an increased concentration of charcoal stripped FBS relative to HEM-1. According to these alternative embodiments, a ROCK inhibitor may be added to HEM-2 during step (ii) upon embryo attachment, i.e. it is not present in HEM-2 until addition upon embryo attachment. Thus, a ROCK inhibitor may be added during step (ii) upon embryo attachment. In a still further embodiment, HEM-3 comprises one or more component of HEM-2 in addition to an increased concentration of charcoal stripped FBS relative to HEM-2. In a further embodiment, HEM-3 comprises HEM-2 in addition to an increased concentration of charcoal stripped FBS relative to HEM-2.

Thus in one aspect, there is provided a human embryo medium 1 (HEM-1) comprising one or more of the following components: a basal medium (Advanced DMEM/F12), B27 supplement, N2 supplement, L-glutamine, antibiotic, N-Acetyl-L-cysteine (NAC), nicotinamide, charcoal stripped FBS, human serum albumin (HSA), hyaluronan, oestrogen (E2), progesterone (P4), insulin-like growth factor 1 (IGF-1), epidermal growth factor (EGF), human prolactin, human Chorionic Gonadotropin (hCG) and human Placental Lactogen (hPL). In one embodiment, HEM-1 comprises a basal medium (Advanced DMEM/F12), B27 supplement, N2 supplement, human serum albumin (HSA), hyaluronan, oestrogen (E2), progesterone (P4), insulin-like growth factor 1 (IGF-1) and epidermal growth factor (EGF). In a further embodiment, HEM-1 comprises a basal medium (Advanced DMEM/F12), B27 supplement, N2 supplement, human serum albumin (HSA), hyaluronan, oestrogen (E2), progesterone (P4), insulin-like growth factor 1 (IGF-1) and epidermal growth factor (EGF), in addition to one or more of: L-glutamine, antibiotic, N-Acetyl-L-cysteine (NAC), nicotinamide, charcoal stripped FBS, human prolactin, human Chorionic Gonadotropin (hCG) and human Placental Lactogen (hPL). In a yet further embodiment, HEM-1 comprises a basal medium (Advanced DMEM/F12), B27 supplement, N2 supplement, L-glutamine, antibiotic, N-Acetyl-L-cysteine (NAC), nicotinamide, charcoal stripped FBS, human serum albumin (HSA), hyaluronan, oestrogen (E2), progesterone (P4), insulin-like growth factor 1 (IGF-1), epidermal growth factor (EGF), human prolactin, human Chorionic Gonadotropin (hCG) and human Placental Lactogen (hPL).

In a further aspect, there is provided a human embryo medium 2 (HEM-2) comprising one or more component of HEM-1 in addition to one or more of: a ROCK inhibitor, glucose, Insulin-Transferrin-Selenium-Ethanolamine (ITS-X), non-essential amino acids, essential amino acids and an increased concentration of charcoal stripped FBS relative to HEM-1. In one embodiment, HEM-2 comprises one or more component of HEM-1 in addition to a ROCK inhibitor, glucose, Insulin-Transferrin-Selenium-Ethanolamine (ITS-X), non-essential amino acids, essential amino acids and an increased concentration of charcoal stripped FBS relative to HEM-1. In an alternative embodiment, HEM-2 comprises one or more component of HEM-1 in addition to glucose, Insulin-Transferrin-Selenium-Ethanolamine (ITS-X), non-essential amino acids, essential amino acids and an increased concentration of charcoal stripped FBS relative to HEM-1. According to this alternative embodiment, a ROCK inhibitor may be added to HEM-2 upon embryo attachment in step (ii). In a further embodiment, HEM-2 comprises: (i) HEM-1 comprising a basal medium (Advanced DMEM/F12), B27 supplement, N2 supplement, human serum albumin (HSA), hyaluronan, oestrogen (E2), progesterone (P4), insulin-like growth factor 1 (IGF-1) and epidermal growth factor (EGF); and (ii) one or more of: a ROCK inhibitor, glucose, Insulin-Transferrin-Selenium-Ethanolamine (ITS-X), non-essential amino acids, essential amino acids and an increased concentration of charcoal stripped FBS relative to HEM-1. In another embodiment, HEM-2 comprises: (i) HEM-1 comprising a basal medium (Advanced DMEM/F12), B27 supplement, N2 supplement, human serum albumin (HSA), hyaluronan, oestrogen (E2), progesterone (P4), insulin-like growth factor 1 (IGF-1) and epidermal growth factor (EGF); and (ii) a ROCK inhibitor, glucose, Insulin-Transferrin-Selenium-Ethanolamine (ITS-X), non-essential amino acids, essential amino acids and an increased concentration of charcoal stripped FBS relative to HEM-1. In another alternative embodiment, HEM-2 comprises: (i) HEM-1 comprising a basal medium (Advanced DMEM/F12), B27 supplement, N2 supplement, human serum albumin (HSA), hyaluronan, oestrogen (E2), progesterone (P4), insulin-like growth factor 1 (IGF-1) and epidermal growth factor (EGF); and (ii) glucose, Insulin-Transferrin-Selenium-Ethanolamine (ITS-X), non-essential amino acids, essential amino acids and an increased concentration of charcoal stripped FBS relative to HEM-1. According to this other alternative embodiment, a ROCK inhibitor may be added to HEM-2 upon embryo attachment in step (ii). In a yet further embodiment, HEM-2 comprises: (i) HEM-1 comprising a basal medium (Advanced DMEM/F12), B27 supplement, N2 supplement, L-glutamine, antibiotic, N-Acetyl-L-cysteine (NAC), nicotinamide, charcoal stripped FBS, human serum albumin (HSA), hyaluronan, oestrogen (E2), progesterone (P4), insulin-like growth factor 1 (IGF-1), epidermal growth factor (EGF), human prolactin, human Chorionic Gonadotropin (hCG) and human Placental Lactogen (hPL); and (ii) one or more of: a ROCK inhibitor, glucose, Insulin-Transferrin-Selenium-Ethanolamine (ITS-X), non-essential amino acids, essential amino acids and an increased concentration of charcoal stripped FBS relative to HEM-1. In a still further embodiment, HEM-2 comprises: (i) HEM-1 comprising a basal medium (Advanced DMEM/F12), B27 supplement, N2 supplement, L-glutamine, antibiotic, N-Acetyl-L-cysteine (NAC), nicotinamide, charcoal stripped FBS, human serum albumin (HSA), hyaluronan, oestrogen (E2), progesterone (P4), insulin-like growth factor 1 (IGF-1), epidermal growth factor (EGF), human prolactin, human Chorionic Gonadotropin (hCG) and human Placental Lactogen (hPL); and (ii) a ROCK inhibitor, glucose, Insulin-Transferrin-Selenium-Ethanolamine (ITS-X), non-essential amino acids, essential amino acids and an increased concentration of charcoal stripped FBS relative to HEM-1. In a still further alternative embodiment, HEM-2 comprises: (i) HEM-1 comprising a basal medium (Advanced DMEM/F12), B27 supplement, N2 supplement, L-glutamine, antibiotic, N-Acetyl-L-cysteine (NAC), nicotinamide, charcoal stripped FBS, human serum albumin (HSA), hyaluronan, oestrogen (E2), progesterone (P4), insulin-like growth factor 1 (IGF-1), epidermal growth factor (EGF), human prolactin, human Chorionic Gonadotropin (hCG) and human Placental Lactogen (hPL); and (ii) glucose, Insulin-Transferrin-Selenium-Ethanolamine (ITS-X), non-essential amino acids, essential amino acids and an increased concentration of charcoal stripped FBS relative to HEM-1. According to this further alternative embodiment, a ROCK inhibitor may be added to HEM-2 upon embryo attachment in step (ii).

In a yet further aspect, there is provided a human embryo medium 3 (HEM-3) comprising one or more component of HEM-2 in addition to an increased concentration of charcoal stripped FBS relative to HEM-2. In one embodiment, HEM-3 comprises: (i) HEM-2 comprising one or more component of HEM-1 in addition to one or more of: a ROCK inhibitor, glucose, Insulin-Transferrin-Selenium-Ethanolamine (ITS-X), non-essential amino acids, essential amino acids and an increased concentration of charcoal stripped FBS relative to HEM-1; and (ii) an increased concentration of charcoal stripped FBS relative to HEM-2. In a further embodiment, HEM-3 comprises: (i) HEM-2 comprising HEM-1 as defined herein in addition to a ROCK inhibitor, glucose, Insulin-Transferrin-Selenium-Ethanolamine (ITS-X), non-essential amino acids, essential amino acids and an increased concentration of charcoal stripped FBS relative to HEM-1; and (ii) an increased concentration of charcoal stripped FBS relative to HEM-2. In an alternative embodiment, HEM-3 comprises: (i) HEM-2 comprising HEM-1 as defined herein in addition to glucose, Insulin-Transferrin-Selenium-Ethanolamine (ITS-X), non-essential amino acids, essential amino acids and an increased concentration of charcoal stripped FBS relative to HEM-1; and (ii) an increased concentration of charcoal stripped FBS relative to HEM-2. According to this alternative embodiment, a ROCK inhibitor may have been added to HEM-2 upon embryo attachment in step (ii). Thus in one embodiment, HEM-3 does not comprise a ROCK inhibitor.

### In Vitro Methods & Uses

According to a further aspect of the invention, there is provided an in vitro method of determining embryo implantation ability and/or window of receptibility to embryo implantation, said method comprising the steps of:
(i) providing the three-dimensional endometrium assembloid defined herein, wherein the endometrial stromal cells and endometrial epithelial cells have been isolated from an endometrial biopsy obtained from a subject;
(ii) stimulating the endometrium assembloid using one or more of oestrogen, progesterone and cyclic AMP (CAMP), thereby rendering the endometrium assembloid receptive to embryo implantation; and
(iii) culturing a human stem cell based blastoid or human blastocyst donated by an IVF patient in the presence of the stimulated endometrium assembloid, thereby allowing the blastoid or blastocyst to implant into the endometrium assembloid,
optionally wherein the blastocyst is isolated from the subject or the blastoid is derived from cells of the subject.

In a further aspect, there is provided a use of the endometrium assembloid defined herein for determining embryo implantation ability and/or window of receptibility to embryo implantation, comprising:
(i) stimulating the endometrium assembloid using one or more of oestrogen, progesterone and cyclic AMP (CAMP), thereby rendering the endometrium assembloid receptive to embryo implantation; and
(ii) culturing a human stem cell based blastoid or human blastocyst donated by an IVF patient in the presence of the stimulated endometrium assembloid, thereby allowing the blastoid or blastocyst to implant into the endometrium assembloid,
wherein the endometrial stromal cells and endometrial epithelial cells have been isolated from an endometrial biopsy obtained from a subject, and
optionally wherein the blastocyst is isolated from the subject or the blastoid is derived from cells of the subject.

In certain embodiments, step (iii) of the in vitro method of determining embryo implantation ability and/or window of receptibility to embryo implantation comprises culturing a human stem cell based blastoid or human blastocyst donated by an IVF patient in the presence of the stimulated endometrium and one or more of oestrogen, progesterone and cyclic AMP (cAMP). In another embodiment, step (ii) of the use of the endometrium assembloid defined herein comprises culturing a human stem cell based blastoid or human blastocyst donated by an IVF patient in the presence of the stimulated endometrium and one or more of oestrogen, progesterone and cyclic AMP (CAMP). In further embodiments, step (iii)/step (ii) comprises culturing in the presence of oestrogen, progesterone and cAMP.

Determining embryo implantation ability is useful during in vitro fertilisation (IVF) treatment for ensuring that the endometrium of a subject, such as a human subject in need of IVF treatment, is receptive to embryo implantation. As mentioned hereinbefore, embryo implantation is a significantly limiting step in pregnancy and thus during IVF treatment, despite advances in the ability to select quality embryos during such treatment. Thus in certain embodiments, the subject is undergoing IVF treatment, or is in need of IVF treatment.

Determining the window of receptibility to embryo implantation is also useful during IVF treatment, as this can vary between subjects/individuals. For example, while it is known that the luteal phase is approximately 14 days in length in humans, this is only an approximate period and may vary between individual subjects.

IVF is a process of fertilisation where an egg is combined with sperm in vitro. The process/treatment involves monitoring and stimulating a woman's ovulatory process, removing an ovum or ova (egg or eggs) from her ovaries and letting sperm fertilise them in a culture medium in a laboratory. After the fertilised egg (zygote) undergoes embryo culture for 2-6 days (e.g. until the cleavage stage or the blastocyst stage), it is transferred into the uterus, with the intention of establishing a successful pregnancy. Therefore, by determining embryo implantation ability for an individual subject, the likelihood of a successful pregnancy following transfer of the embryo into the uterus of said subject can be determined. Furthermore, by determining the window of receptibility to embryo implantation for an individual subject, the optimum time for transfer into the uterus can be identified for said subject. In combination, determining embryo implantation ability and window of receptibility to embryo implantation for an individual subject can be used to provide the highest likelihood of successful pregnancy following transfer and determine said likelihood.

Thus in a certain aspect, there is provided the endometrium assembloid defined herein for use in a method of in vitro fertilisation (IVF) treatment. In a further aspect, there is provided the endometrium assembloid defined herein for use in IVF, such as IVF treatment. In another aspect, there is provided a use of the endometrium assembloid in a method of IVF treatment. In a yet further aspect, there is provided a use of the endometrium assembloid defined herein in IVF, such as IVF treatment. In a yet other aspect, there is provided a method of IVF, such as IVF treatment, comprising the endometrium assembloid defined herein. In certain embodiments, said IVF treatment is for the treatment of infertility. In a further embodiment of these aspects of the invention, the endometrium assembloid is for use in determining embryo implantation ability and/or window of receptibility to embryo implantation for a subject during IVF, IVF treatment and/or the method of IVF treatment. In a yet further embodiment, the IVF, IVF treatment and/or method of IVF treatment comprises a step of determining embryo implantation ability and/or window of receptibility to embryo implantation for a subject.

In a particular embodiment, the endometrial stromal cells and endometrial epithelial cells used to generate the three-dimensional endometrium assembloid defined herein are obtained from a subject undergoing IVF treatment or are obtained from a subject in need of IVF treatment. Thus in one embodiment, the endometrial stromal cells and endometrial epithelial cells have been isolated from an endometrial biopsy obtained from a subject undergoing IVF treatment or obtained from a subject in need of IVF treatment. In a further embodiment, the endometrial biopsy has been obtained from the subject concurrently with egg removal/collection or isolation. Thus in a yet further embodiment, the egg is isolated from the subject, i.e, wherein the subject is the same subject from which the endometrial biopsy has been obtained and is undergoing IVF treatment, or is in need of IVF treatment.

In one embodiment, the in vitro method of determining embryo implantation ability and/or window of receptibility to embryo implantation method comprises the step of: (ii) stimulating the endometrium assembloid using one or more of oestrogen, progesterone and cAMP. Such hormonal stimulation leads to decidualization of the endometrium assembloid as described hereinbefore, thereby rendering the endometrium assembloid receptive to embryo implantation. In one embodiment, the endometrium assembloid is receptive to implantation of a blastoid in vitro. In another embodiment, the endometrium assembloid is receptive to blastocyst implantation in vitro. As demonstrated herein, following stimulation of the endometrium assembloid using oestrogen, progesterone and cAMP both stem cell-based blastocyst models (blastoids) and human blastocysts from an IVF patient are able to implant into the endometrium assembloid described herein, with the trophoblast able to fully invade the endometrial scaffold and reach the stromal compartment and blastoids/blastocysts orientated with the polar region of the trophectoderm directly towards the surface of the endometrial scaffold prior to implantation. This is in contrast to previously known two-dimensional culture models (Deglincerti et al. (2016); and Shahbazi et al. (2016)), where embryos are seen to adopt a flattened structure and loose native morphology. Furthermore, implantation into the endometrium assembloid is marked by the contraction of the blastocoel cavity and the formation of a visible patch at the site of adhesion of the embryo onto the surface, and after implantation of the embryo into the endometrium assembloid, physiological growth of the embryo can be observed, as demonstrated by formation of a clear expanded yolk sac cavity that was demarcated by flat, dispersed SOX17+ primary yolk sac endoderm cells. In vitro implantation of the embryo into the assembloid is concomitant with an increase in secretions of human chorionic gonadotropin (hCG), the primary biochemical marker used in the recognition of a clinical pregnancy, and by 10 d.p.f., embryos fully penetrate the epithelial layer and reach the deep stroma of the endometrial assembloid, disappearing from view from the surface. Thus, the endometrium assembloid defined herein allows natural/physiological implantation of an embryo or blastoid/blastocyst and supports the natural/physiological three-dimensional growth of said embryo or blastoid/blastocyst in vitro.

As will be readily appreciated by those skilled in the art, references herein to “blastoid” refer an embryoid, a stem cell-based embryo model which, morphologically and transcriptionally resembles the early, pre-implantation, mammalian conceptus (the blastocyst). The first blastoids were created by combining mouse embryonic stem cells and mouse trophoblast stem cells. Upon in vitro development, blastoids generate analogues of the hypoblast, thus comprising analogues of the three founding cell types of the conceptus (epiblast, trophoblast and hypoblast cells), and recapitulate aspects of implantation. However, blastoids do not display the capacity to support the development of a foetus and are therefore not considered as an embryo but as a model. Thus in certain embodiments of the invention, the methods described herein do not comprise the destruction of a human embryo. Compared to other stem cell-based embryo models (e.g. gastruloids), blastoids model the preimplantation stage and the integrated development of the conceptus including the embryo proper and the two extraembryonic tissues (trophectoderm and hypoblast). As demonstrated herein, blastoids initiate apposition to the surface of the endometrium assembloid, retaining a clear expanded blastocoel cavity, and were capable of implantation into the assembloid, penetrating the endometrial epithelium and invading into the endometrial stromal compartment.

As will also be readily appreciated, references herein to “blastocyst” refer to the structure formed in the early embryonic development of mammals. In one embodiment, the blastocyst cultured in the methods described herein is a primary human blastocyst, such as a blastocyst from a subject undergoing IVF as defined hereinbefore. Such blastocysts from a subject undergoing IVF may be surplus blastocysts from the IVF procedure. The blastocyst possesses an inner cell mass also known as the embryoblast which subsequently forms the embryo, and an outer layer of cells called the trophectoderm. The trophectoderm surrounds the inner cell mass and a fluid-filled cavity known as the blastocoel. In the late blastocyst the trophectoderm is known as the trophoblast, and this gives rise to the chorion that surrounds the embryo. In humans, blastocyst formation begins about five days after fertilisation when a fluid-filled cavity opens up in the morula, the early embryonic stage of a ball of 16 cells. The blastocyst has a diameter of about 0.1-0.2 mm and comprises 200-300 cells following rapid cleavage (cell division). About seven-eight days after fertilisation, the blastocyst undergoes implantation, embedding into the endometrium of the uterine wall as described hereinbefore, after which it will undergo further developmental processes, including gastrulation. Embedding of the blastocyst into the endometrium requires hatching from the zona pellucida, which prevents adherence to the fallopian tube as the pre-embryo makes its way to the uterus.

Demonstrated herein is the ability of the endometrium assembloid to support the longer term growth of human embryos until at least 14 d.p.f., a key stage of development that has remained largely unexplored to date due to difficulties in maintaining embryo viability is culture over a prolonged time span. Specifically, deep invasion and integration of the conceptus inside the endometrium is observed as demonstrated by the fading after 14 d.p.f. of a dark shadow initially seen at the area of the implanting embryo within the scaffold, and the intermingling of GATA3+ trophoblast cells with endometrial cells is seen. 14 d.p.f. embryos implanted into the assembloid show advanced morphological features, including appearance of the bilaminar disc comprised of a layer of OCT4+ epiblast cells adjacent to a layer of SOX17+ hypoblast cells, with clear expanded pro-amniotic and primary yolk sac cavities on either side of the disc. The emergence of the extraembryonic mesenchymal lineage is also seen as indicated by the presence of VIM+ cells overlaying the hypoblast and lining the outer surface of the yolk sac cavity. Thus, the endometrium assembloid defined herein supports the long term development of embryos to develop advanced morphological features closely recapitulating histological samples of in vivo implantation sites at Carnegie Stage 6 (Hertig, Rock & Adams (1956) Am. J. Anat., 98(3):435-493, doi: https://doi.org/10.1002/aja.1000980306). Still further, following implantation into the endometrium assembloid the embryo at 14 d.p.f. is capable of forming large outgrowths characterised by the extensive proliferation and differentiation of trophoblast cells, with these invading the endometrium and embedding within the stroma, consistent with the deep interstitial invasiveness of the human conceptus. Trophoblastic outgrowths into the assembloid are characterised by single-nucleated GATA3+ cytotrophoblast cells and by the formation of plate-like assemblies of multi-nucleated cells at the leading edge of the invading trophoblast cells which confirms syncytial transformation of the trophoblast cells. The start of the embryo-maternal interface for the establishment of the early placenta as described in historical anatomical sections from the Carnegie collection (but not previously observed in culture) is also seen in 14 d.p.f. embryos implanted into the present assembloid, as demonstrated herein by the presence of uninuclear KRT7+ trophoblast cells extending as strands through the syncytium to the margin of the implantation site which represent the forerunners of the cytotrophoblast cell columns and characterise the transformation of the syncytial trabeculae into primary villi, which herein show signs of proliferation, branching and the initiation of focal anchorage contacts between the conceptus and decidualised stromal cells.

In one embodiment, the result or read-out for determining embryo implantation ability and/or window of receptibility to embryo implantation in the methods defined herein is embryo or blastoid/blastocyst implantation. In a further embodiment, the result or read-out is the period of time taken for embryo or blastoid/blastocyst implantation. In a yet further embodiment, the result or read-out is the amount or level (e.g. the depth) of implantation of the embryo or blastoid/blastocyst.

In a yet further aspect of the invention, there is provided a method of screening for an embryo implantation-modulating agent, said method comprising the steps of:
(i) providing the three-dimensional endometrium assembloid defined herein;
(ii) stimulating the endometrium assembloid using one or more of oestrogen, progesterone and cyclic AMP (CAMP) in the presence and/or absence of a test agent; and
(iii) culturing a human stem cell based blastoid or human blastocyst donated by an IVF patient in the presence of the stimulated endometrium assembloid and one or more of oestrogen, progesterone and cyclic AMP (CAMP),
wherein a difference between the implantation of the blastoid or blastocyst into the endometrium assembloid stimulated in the presence of the test agent and the implantation of the blastoid or blastocyst into the endometrium assembloid stimulated in the absence of the test agent is indicative of the implantation-modulating effect of said agent.

It will be appreciated that according to this aspect of the invention, the test agent may comprise any compound, treatment, condition or process which may enhance, increase or accelerate the implantation of an embryo or blastoid/blastocyst into the endometrium assembloid, or alternatively may diminish, decrease or decelerate implantation. Thus in one embodiment, the test agent enhances, increases or accelerates implantation. In an alternative embodiment, the test agent diminishes, decreases or decelerates implantation. In a further embodiment, the test agent prevents implantation. In another embodiment, the test agent increases the period of time taken for an embryo or blastoid/blastocyst to implant. In an alternative embodiment, the test agent decreases the period of time taken for an embryo or blastoid/blastocyst to implant. In a yet further embodiment, the test agent decreases the amount or level (e.g. the depth) of implantation. In another embodiment, the test agent increases the amount or level (e.g. the depth) of implantation.

Thus in certain embodiments, the difference between the implantation of the blastoid or blastocyst (e.g. an embryo) is determined between implantation into the endometrium assembloid stimulated in the presence of the test agent and implantation into the endometrium assembloid stimulated in the absence of the test agent. In a further embodiment, the difference between implantation of the blastoid or blastocyst (e.g. an embryo) is between implantation into an endometrium assembloid generated from endometrial biopsies taken from a “normal” or “healthy” tissue or subject and implantation into an endometrium assembloid generated from endometrial biopsies taken from a “diseased” tissue or subject, or from endometrial biopsies taken from tissue suspected to be disease or otherwise abnormal.

In certain embodiments of the in vitro methods, uses, methods of treatment and endometrium assembloid for use defined herein, stimulating the endometrium assembloid comprises using oestrogen for between 1 and 7 days, followed by using oestrogen, progesterone and CAMP for between 2 and 10 days, such as 8 days. In a further embodiment, stimulating comprises using oestrogen for 4 days, followed by oestrogen, progesterone and CAMP for between 2 and 10 days, such as 8 days. In a yet further embodiment, stimulating comprises using oestrogen for 4 days, followed by using oestrogen, progesterone and CAMP for between 1 and 6 days. In a particular embodiment, stimulating comprises using oestrogen for 4 days, followed by using oestrogen, progesterone and CAMP for between 1 and 4 days. In another embodiment, stimulating comprises using oestrogen for between 1 and 7 days, followed by using oestrogen, progesterone and CAMP for between 1 and 6 days. In a still further embodiment, stimulating comprises using oestrogen for between 1 and 7 days, followed by using oestrogen, progesterone and CAMP for between 1 and 4 days.

In further embodiments, culturing the blastoid or blastocyst in the presence of the stimulated endometrium assembloid and hormones is performed between 1 and 14 days after stimulating. In a yet further embodiment, said culturing is performed between 2 and 10 days, such as 8 days after stimulating. In a still further embodiment, said culturing is performed between 1 and 7 days after stimulating. As will be appreciated, culturing the blastoid or blastocyst in the presence of the stimulated endometrium assembloid is also performed in the presence of hormones, in particular in the presence of oestrogen, progesterone and cAMP.

However, as will be understood by those skilled in the art, the hormones referred to herein (namely oestrogen, progesterone and cAMP) may be replaced with any synthetic or naturally occurring analogue thereof. Examples of oestrogens and analogues thereof include, without limitation, bioidentical oestradiol, natural conjugated oestrogens, synthetic steroidal oestrogens like estradiol and its analogue ethinylestradiol, and synthetic nonsteroidal oestrogens like diethylstilbestrol. Examples of progesterone and analogues thereof include, without limitation, the synthetics medroxyprogesterone acetate and norethisterone, progesterone ethers including quingestrone (progesterone 3-cyclopentyl enol ether) and progesterone 3-acetyl enol ether, as well as derivatives of the groups of retroprogesterone, 17α-hydroxyprogesterone, 17α-methylprogesterone, and 19-norprogesterone, which include dydrogesterone, medroxyprogesterone acetate, medrogestone and promegestone, respectively. Examples of CAMP analogues include, without limitation, small molecule analogues such as 6-Bnz-CAMP among others.

Unless defined otherwise, all technical and scientific terms used herein have the meaning commonly understood by a person skilled in the art to which this invention belongs. As used herein, the term “about” when used herein includes up to and including 10% greater and up to and including 10% lower than the value specified, suitably up to and including 5% greater and up to and including 5% lower than the value specified, especially the value specified. The term “between” as used herein includes the values of the specified boundaries.

Throughout the specification and the claims which follow, unless the context requires otherwise, the word “comprise”, and variations thereof such as “comprises” and “comprising”, will be understood to imply the inclusion of a stated integer, step, group of integers or group of steps but not to the exclusion of any other integer, step, group of integers or group of steps.

In addition, as used herein and the appended claims, the singular forms “a”, “an” and “the” include plural referents unless the content clearly dictates otherwise. Thus, for example reference to “a tissue isolate/glandular element” includes two or more such isolates, or reference to “an endometrium assembloid” includes two or more such assembloids and the like.

It will be understood that all embodiments described herein may be applied to all aspects of the invention and vice versa, and such combinations would be readily apparent from the description provided herein and to those skilled in the art.

Other features and advantages of the present invention will be apparent from the description provided herein. It should be understood, however, that the description and the specific examples while indicating preferred embodiments of the invention are given by way of illustration only, since various changes and modifications will become apparent to those skilled in the art. The invention will now be described using the following, non-limiting examples:

### EXAMPLES

### Example 1: Modelling the Cytoarchitecture of the Human Endometrium

It was hypothesised that recreating a functional maternal environment would be essential to enable the human blastocyst to undergo implantation in vitro. To this end, a three-dimensional culture model (assembloid) was generated which includes the major cell types and tissue features of the mature endometrium, with the goal to recreate the receptive maternal interface necessary for implantation. Based on previously developed endometrial models (Li et al. (2022) Reprod. Biol. Endocrinol., 20(120), doi: https://doi.org/10.1186/s12958-022-00973-8; Weimar et al. (2013) Reprod. Biomed. Online, 27(5):461-476, doi: https://doi.org/10.1016/j.rbmo.2013.08.002; Ojosnegros et al. (2021) Hum. Reprod. Update, 27(3):501-530, doi: https://doi.org/10.1093/humupd/dmaa054; and Popovic & Chuva de Sousa Lopes (2022) Placenta, 125:36-46, doi: https://doi.org/10.1016/j.placenta.2022.01.006), it was decided to construct a system which comprises three key constituents: a stromal compartment to permit penetration and invasion of the embryo in a physiological three-dimensional substratum; an outer luminal epithelial layer, which acts as site of anchorage to support initial embryo apposition and adhesion; and finally a functional secretory glandular endometrium able to provide histotrophic nourishment to support embryo growth during implantation.

Firstly, a tuneable hydrogel backbone was employed, composed of dextran polymers and cyclodextrin crosslinkers. This enabled the elastic modulus of the hydrogel to be fine-tuned to close to 250 Pa, to recapitulate the tissue stiffness of the natural endometrium, as measured during the mid-secretory phase of the menstrual cycle (Abbas et al. (2019)). Another feature incorporated into the system was tissue degradability. To achieve this, a crosslinker containing matrix metalloprotease cleavable sites was employed. This feature is vital to allow penetration and invasion by the trophoblast cells during the process of implantation, ensuring burrowing of the embryo into the stromal layer through active tissue degradation.

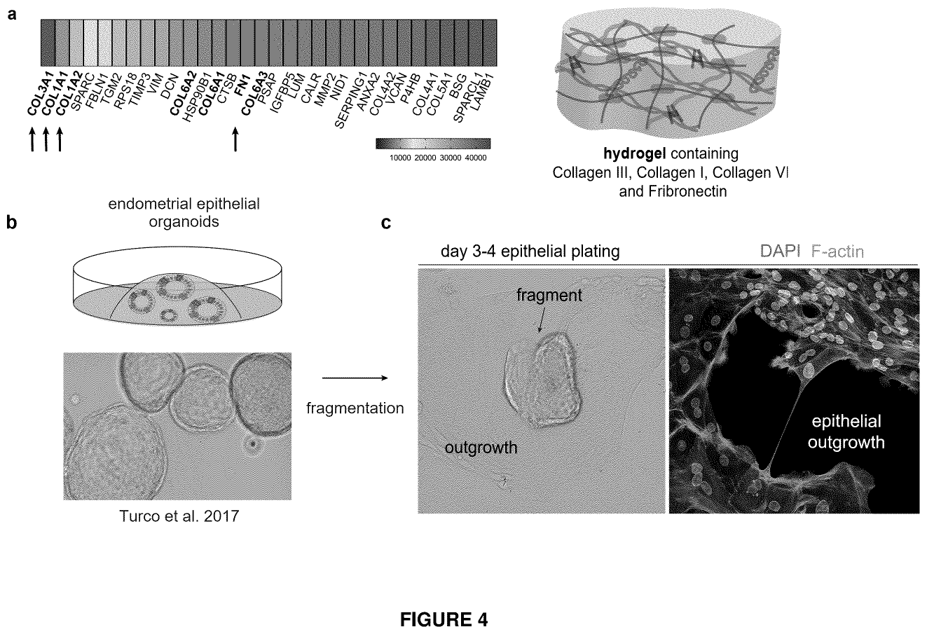

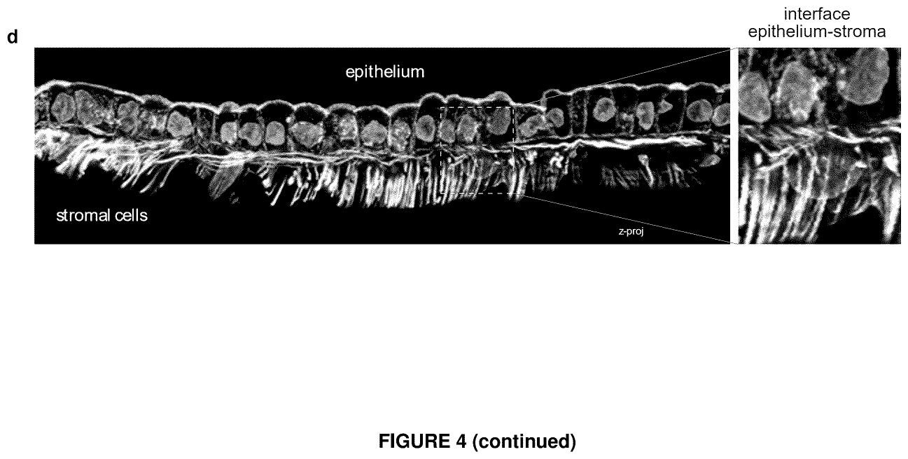

Despite the biochemically defined and synthetic nature, the hydrogel lacked any component which could promote functional interactions between cells and the scaffold. To closely mimic the composition of the extracellular matrix (ECM) of the natural endometrium (Aplin, Charlton & Ayad (1988)), the major ECM transcripts expressed in vivo in the endometrium during the mid-secretory phase were analysed by RNA-seq (data not shown). Among the most highly expressed candidates, collagen III, I, VI and fibronectin were selected as structural components able to form complex matrix networks. In contrast to previous models based on less-defined ECM extracts, such as Matrigel (Arnold et al. (2002); and Buck et al. (2015)), or single defined components such as collagen I Bentin-Ley et al. (1994)) or Fibrin (Wang et al. (2012)), it was decided to incorporate relative ratios of collagen III, I, VI and fibronectin into the gel backbone, with the goal to better mimic the biochemical composition of the native endometrium (FIG. 4).

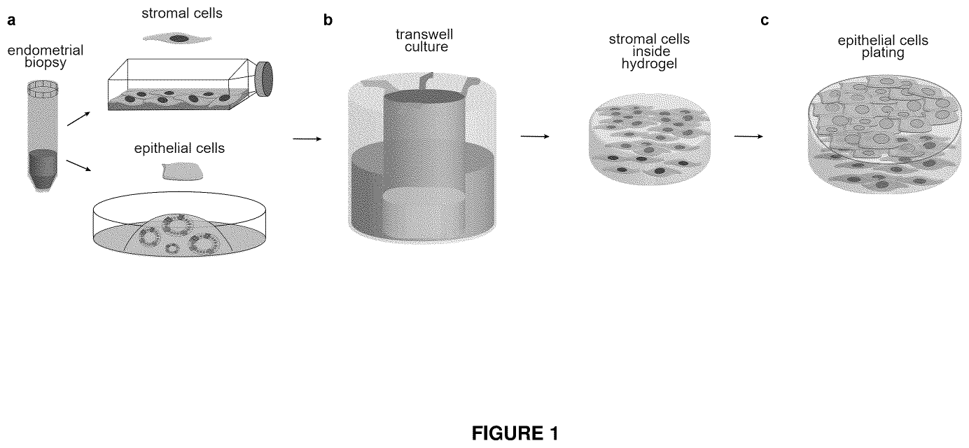

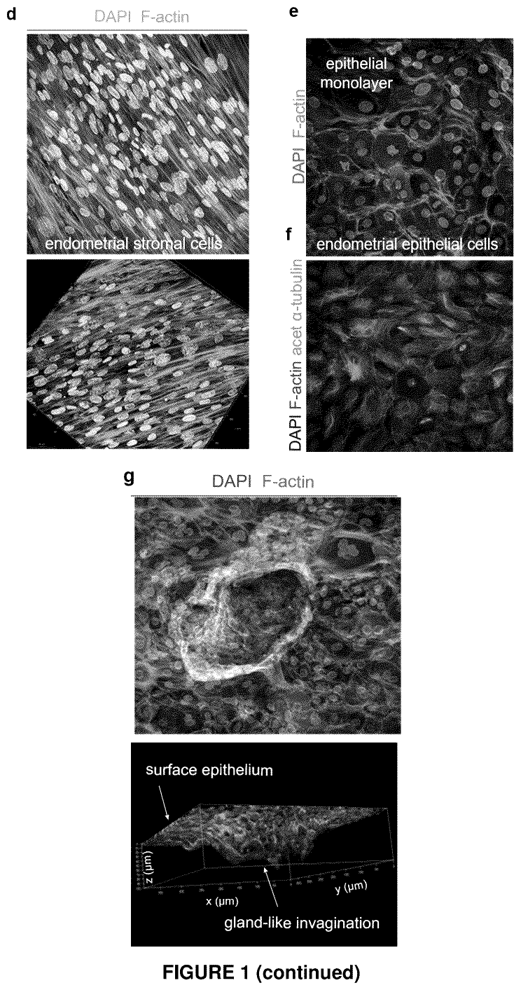

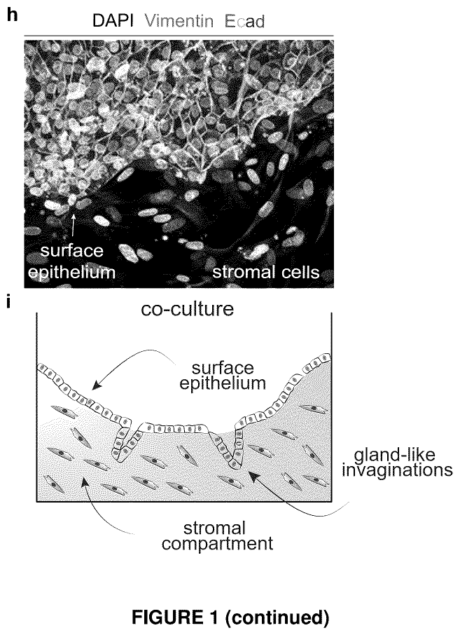

As a first step, primary stromal cells were isolated from endometrial biopsies of fertile patients based on the protocol of Michalski et al. ((2018) J. Vis. Exp., doi: https://dx.doi.org/10.3791/57684; FIG. 1A) and further details are provided in the Materials and Methods section. Stromal cells were incorporated into the hydrogel matrix exploiting the slow gelation properties of the dextran polymers and cultured inside a transwell system in three-dimensions (FIG. 1B). Within 4 to 6 days of culture, stromal cells elongated extensively, giving rise to a highly ordered interconnected network throughout the entire gel depth (FIG. 1D). To generate the epithelial compartment, epithelial cells were isolated from endometrial biopsies and propagated using a previously established organoid culture system (Turco et al. (2017); FIG. 1A, FIG. 4B). Fragments from organoids were then plated on top of the hydrogel containing the stromal cells, to induce tissue assembly by sequential layering deposition (FIG. 1C). Within 3-4 days from seeding, epithelial outgrowths originating from the organoid fragments could be observed (FIG. 4B), eventually forming a single continuous monolayer covering the entire surface of the well (FIG. 1E). Similar to the luminal epithelium, which in vivo represents the first site of contact for human embryo implantation, this surface-located monolayer was particularly rich in acetylated α-tubulin (FIG. 1F). Surprisingly, the epithelium was highly heterogenous in shape, displaying numerous invaginations throughout the entire surface of the well (FIG. 1G), which resemble anatomically the deep-reaching uterine glands (Yamaguchi et al. (2021) iScience, 24(4):102258, doi: https://doi.org/10.1016/j.isci.2021.102258).

These data support the notion that human endometrial epithelial cells isolated from organoids retain the bi-potential capability to give rise to both luminal and glandular lineages (Turco et al. (2017); and Negami & Tominaga (1989) Hum. Reprod., 4(6):620-624, doi: https://doi.org/10.1093/oxfordjournals.humrep.a136954), and are able to self-organise into a superficial luminal epithelium continuous to glandular-like invaginations on a stromal compartment in the present endometrium assembloid culture (FIG. 11, FIG. 4D).

### Example 2: Responsiveness to Hormonal Stimulation and Features of Endometrial Receptivity

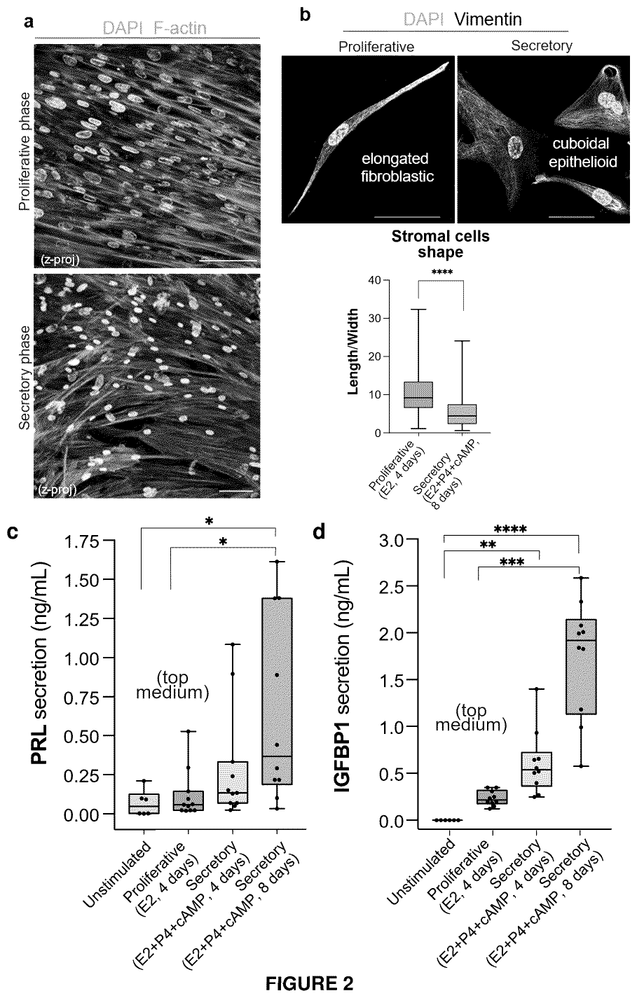

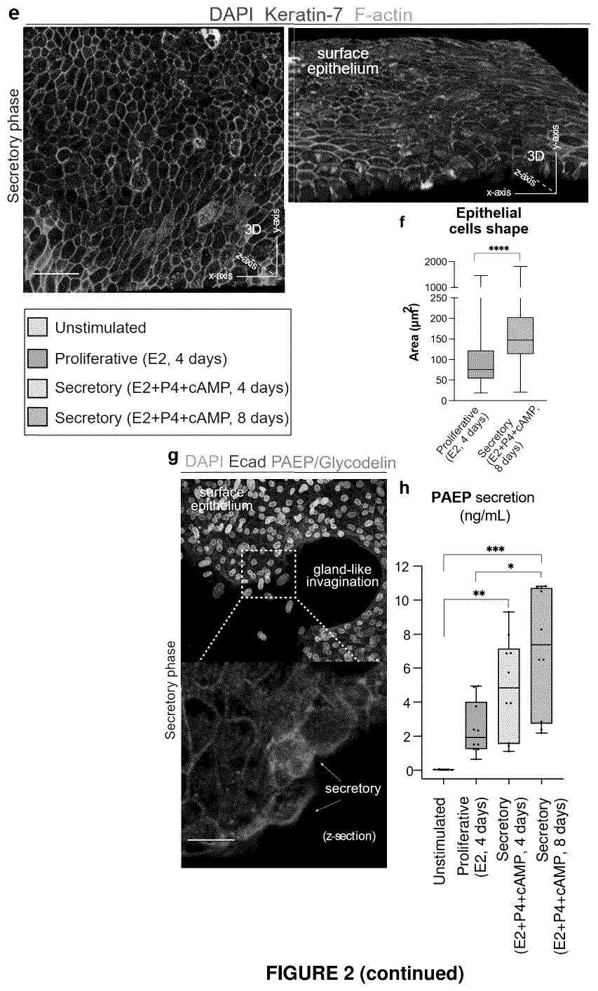

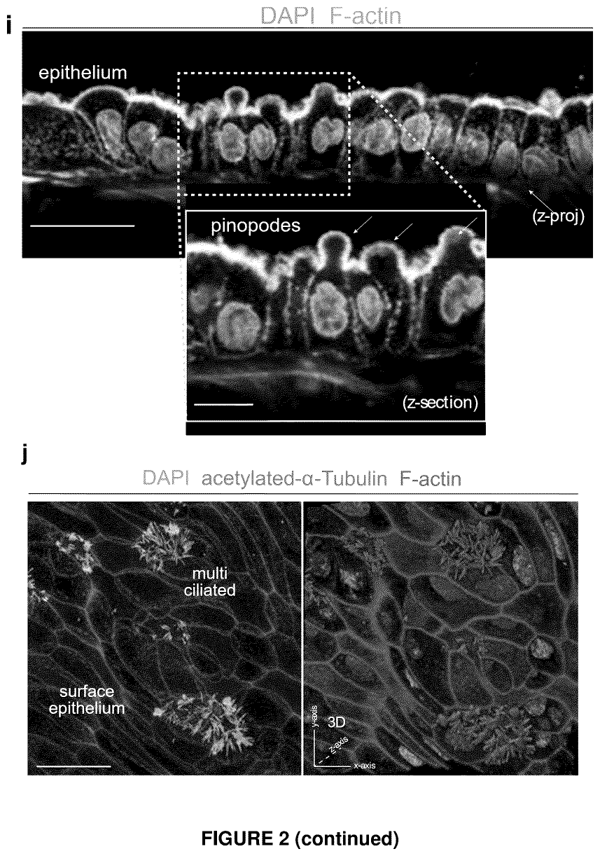

Implantation in vivo occurs within a short time frame, known as the window of implantation, between day 20 and 24 of the menstrual cycle, corresponding to the mid-secretory phase (Cha, Sun & Dey (2012) Nat. Med., 18(12):1754-1767, doi: https://doi.org/10.1038/nm.3012). At this stage, the uterine tissue becomes highly receptive for embryo implantation, undergoing extensive cellular remodelling under the influence of maternal hormones oestrogen (E2) and progesterone (P4; Gellersen & Brosens (2014)). To test whether the newly engineered endometrium assembloid was capable to respond to hormonal stimulation and to adopt tissue features of the receptive window of implantation, E2 was firstly introduced to mirror the natural peak of E2 of the proliferative phase of the cycle, followed by E2, P4 and CAMP to induce differentiation towards the mid-secretory phase (mid-luteal phase). Upon hormonal stimulation, stromal cells underwent extensive morphological transformations and reorganisation of the actin cytoskeleton, as shown by the shift from an elongated fibroblast-like morphology, during the proliferative phase (E2 stimulation), to a rounded epithelial-like configuration during the secretory phase (E2, P4 and CAMP stimulation; FIGS. 2A and 3B, FIG. 5A).

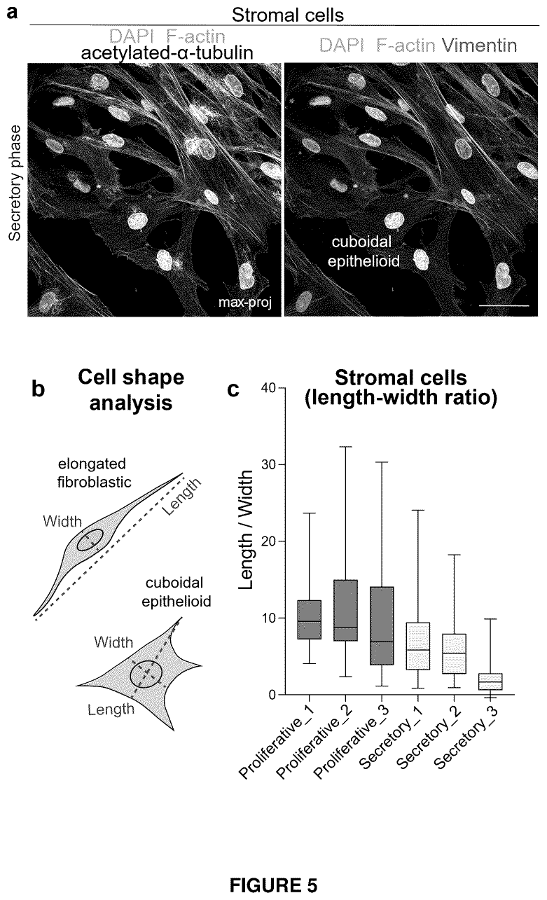

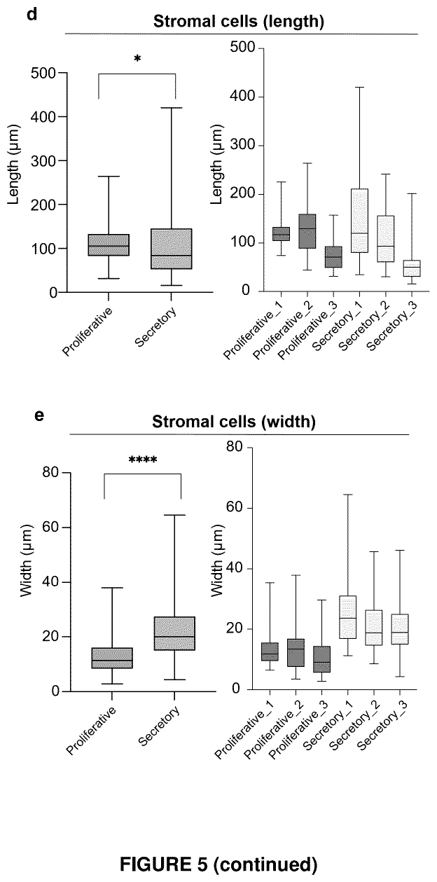

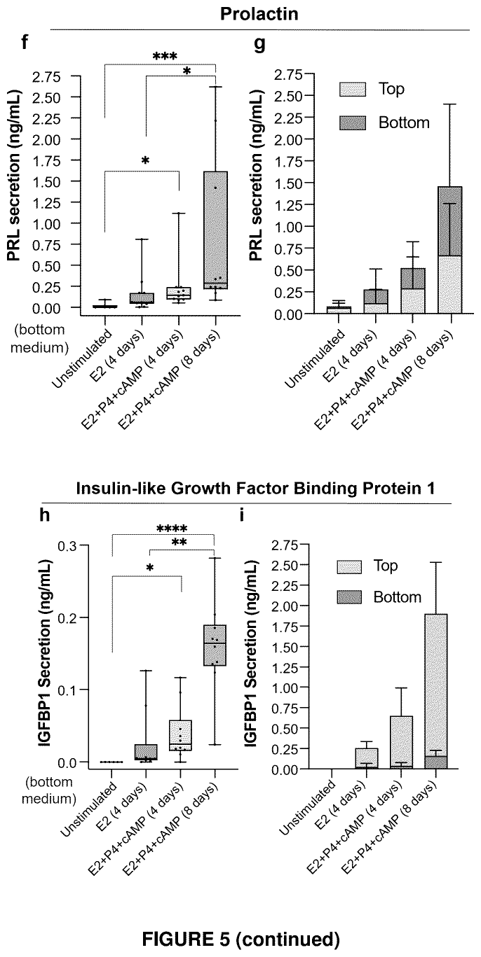

To quantify the level of tissue decidualization achieved in culture, the cell shape changes between the proliferative (E2 stimulation) and secretory phase (E2, P4 and cAMP stimulation) were quantified by manual morphometric analysis (FIG. 5B). This analysis revealed that the shift from elongated to epithelioid morphology (FIG. 5C) was mainly driven by a significant increase in width of stromal cells during the secretory phase (FIG. 5E), while a decrease in length was also noticeable (FIG. 5D). Alongside cytoskeletal rearrangements, hormonal supplementation also induced the acquisition of a secretory-like phenotype in stromal cells, as confirmed by the progressive increase in prolactin (PRL; FIG. 2C, FIGS. 5F and 5G) and insulin-like growth factor binding protein 1 (IGFBP1; FIG. 2D, FIGS. 5H and 51) detected in culture media. The morphological changes observed together with acquisition of secretory capabilities upon hormonal stimulation are central hallmarks reminiscent of the process of tissue decidualization of the human endometrium during the mid-secretory phase (Gellersen & Brosens (2014)).

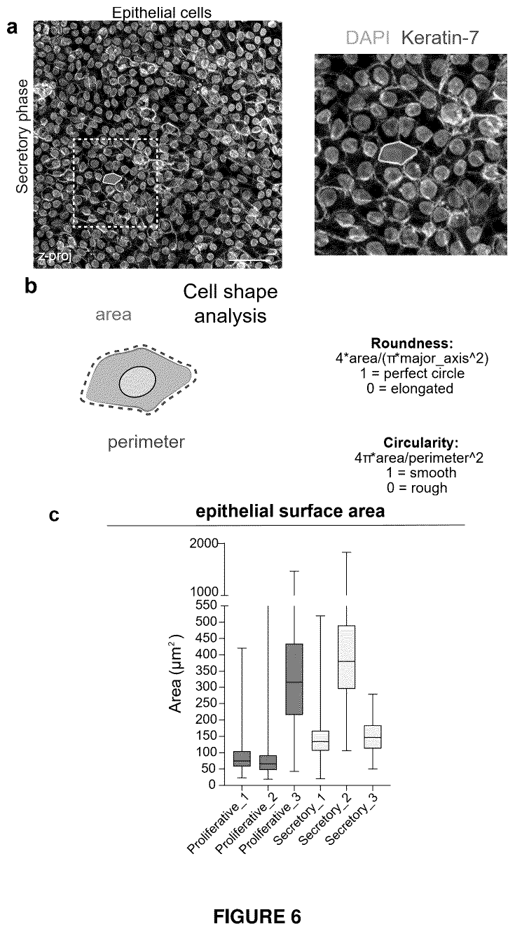

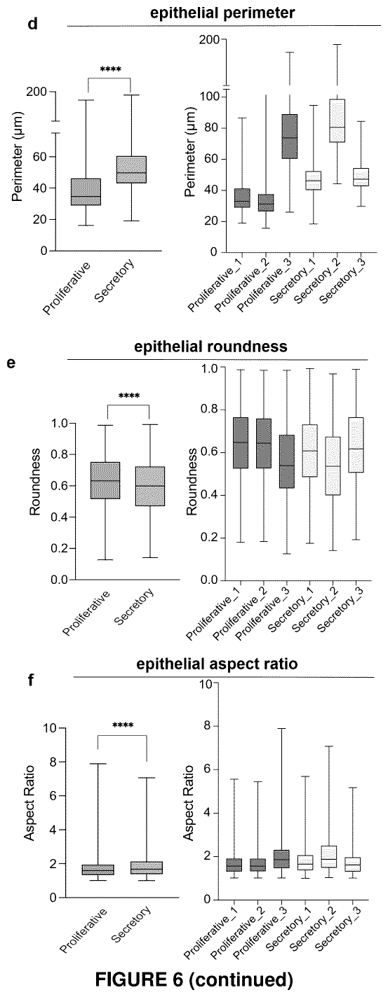

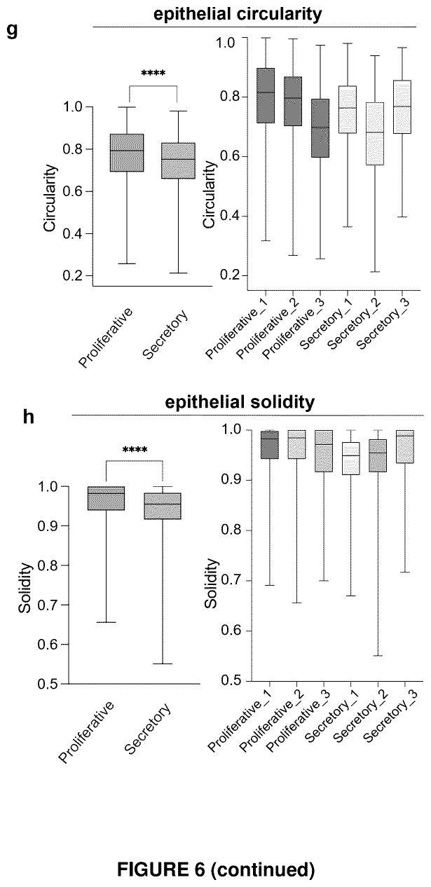

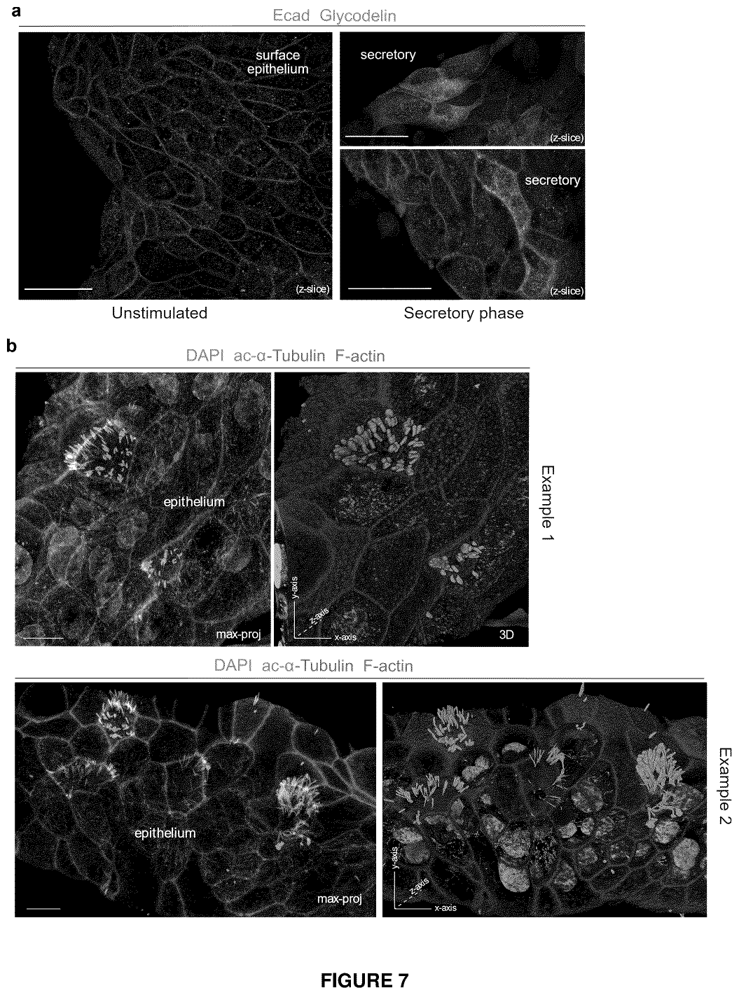

Endometrial cells of the surface epithelium also undergo morphological remodelling during the secretory phase (FIGS. 6A and 6B). Upon hormonal stimulation, epithelial cells increase in apical cell surface area (FIGS. 2E and F, FIG. 6C) and perimeter (FIG. 6D), adopt a more elongated morphology as shown by decreased roundness and increased aspect ratio (FIGS. 6E and 6F), and display irregular borders due to decrease in circularity and solidity (FIGS. 6G and 6H) compared to a smoother and circular morphology during the proliferative phase. The observed changes were consistent with the acquisition of a secretory phenotype, as shown by the progressive increase in glycodelin (PAEP) secretions (FIGS. 7A and 7B). Glycodelin is a major constituent of the secretions produced by the endometrial glands which are known collectively as the “uterine milk” (Burton, Cindrova-Davies & Turco (2020); and Burton et al. (2002)). Within the epithelium it was observed that cells at the border of the invaginations were positive for glycodelin (FIG. 2G), supporting the glandular nature of the observed self-organising epithelial invaginations. The glandular epithelium in the present system releases secretions directly into the medium, as confirmed by the progressive increase of glycodelin detected in the top well (FIG. 2H). Direct access of the embryo to these glandular secretions is an essential requisite which ensures histotrophic nourishment of the conceptus during most of the first human trimester, before the haemochorial placenta becomes fully functional (Burton, Cindrova-Davies & Turco (2020); and Burton et al. (2002)).

Cross section analysis and scanning electron microscopy confirmed the formation of multiple pinopodes on the apical surface of the epithelium (FIG. 2I), which appear in vivo to be directly involved in the adhesion of the blastocyst to the endometrial surface, and have been proposed to act as primary sites of anchorage for embryo apposition and adhesion (Quinn et al. (2020); and Bentin-Ley et al. (1999)). Within the epithelium, the presence of multiple ciliated cells, displaying either a single cilium or multiple cilia, could be detected as identified by acetylated α-tubulin (FIG. 2J).

Taken together, both the stromal and epithelial cells of the endometrium assembloid defined herein respond to maternal hormonal stimulation and adopt key features of endometrial receptivity, reminiscent of the window of implantation during the mid-secretory phase of the menstrual cycle.

### Example 3: In Vitro Implantation of Human Embryo into the Endometrium Mimics Human Embryo Development

Recent advances in culture conditions have enabled the possibility to grow human blastocysts in vitro beyond 5 d.p.f. However, current models are limited to culture of human embryos either on the surface of plastic substrata in 2D or inside undefined 3D platforms such as Matrigel, failed to recapitulate the in vivo conditions of the uterine environment. Devoid of any maternal tissue, embryos develop in isolation outside their biological environment, precluding the possibility to accurately study the process of implantation.

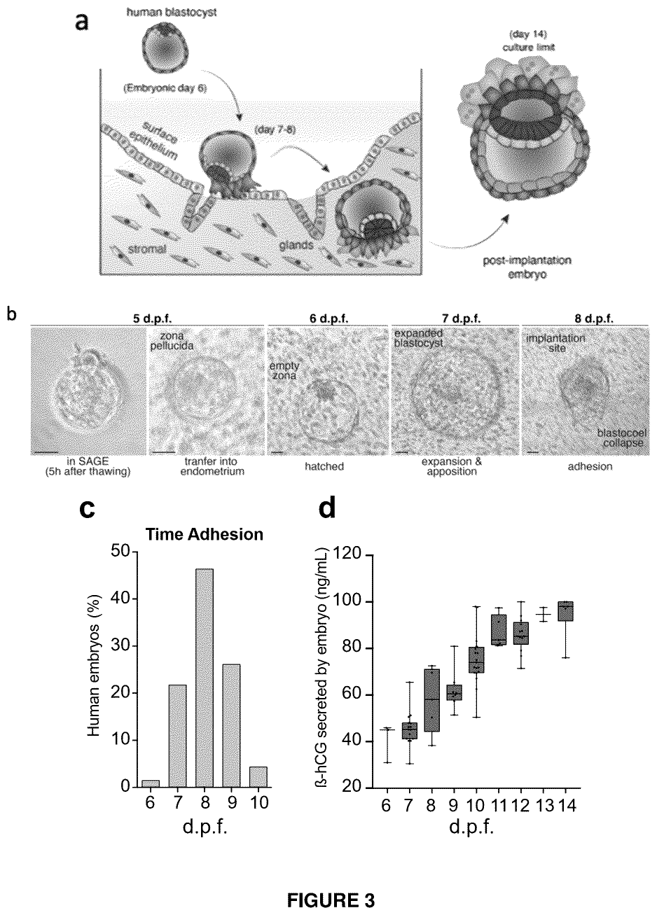

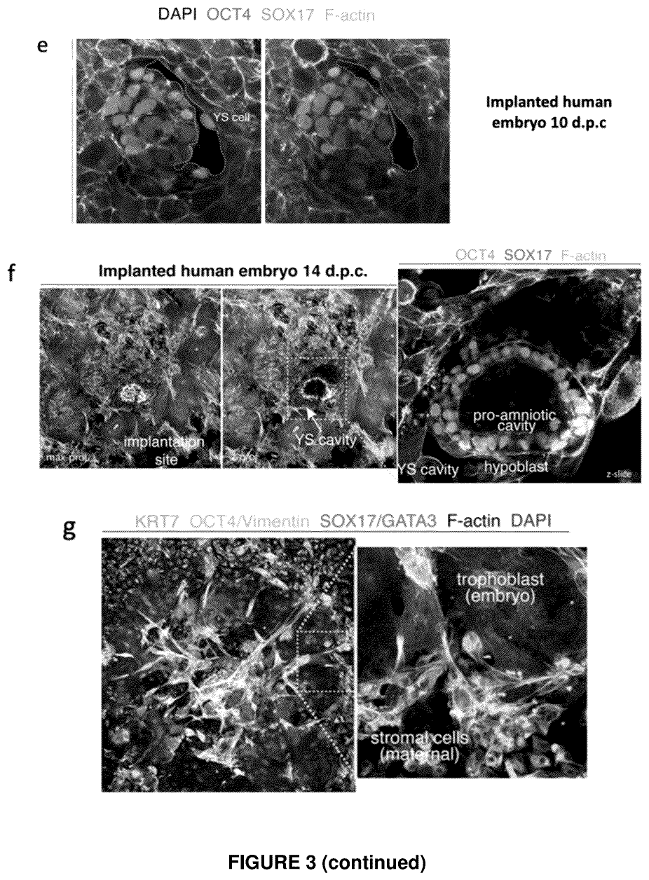

The finding that our endometrial system is able to recapitulate receptive features of the mid-secretory phase led us to ask whether this environment could be competent to permit implantation of the human embryo in vitro. We therefore transferred supernumerary human blastocysts, donated by patients undergoing IVF, inside the endometrial culture (FIG. 3A), synchronising hormonal stimulation towards the receptive window of implantation. Blastocysts at 5 d.p.f. were allowed to hatch naturally from zona pellucida, a process which occurred within 24 h from the transfer (FIG. 3B). Upon hatching, the blastocoel cavity underwent significant expansion, growing 4 times in size over a period of 48-72 h. As previously reported, we confirmed that blastocysts orient upside-down, with the polar region of the trophectoderm facing the surface of the luminal endometrium, while retaining a fully expanded blastocoel morphology. Following this initial phase of apposition, we found that adhesion to the endometrial surface occurred at 8 d.p.f. in the majority of cases (46%), despite significant variabilities existed in the time of adhesion between embryos (FIGS. 3B-3C).

Upon adhesion, the blastocoel cavity contracted, and a clear implantation site became visible on the surface of the endometrium. We noted that the increase over time of the area of implantation mirrored closely the steady increase in human chorionic gonadotropin (hCG) secreted by the embryo into the culture medium (FIG. 3E), supporting physiological development of the embryo in these culture settings.

We found that the trophoblast actively invaded the endometrial scaffold, reaching the deeper stromal layer by 10 d.p.f. In contrast to previous in vitro cultures, where embryos became flattened loosing native morphology, the engineered endometrial tissue was able to support growth of the human embryo in 3D, as indicated by formation of an expanded yolk sac cavity (FIG. 3E). This was demarcated by flat and dispersed SOX17+ cells which contrasted to the columnar and highly packed morphology of the SOX17+ cells of the hypoblast layer, lining the basal domain of the epiblast (OCT4+) (FIG. 3E).

To gain insights into early post-implantation, we decided to further grow implanted embryos until 14 d.p.f., a key stage of development that has remained largely unexplored due to the difficulty to maintain embryos viable in culture over such a prolonged time span. At this stage, the borders of the implantation area become largely elusive under brightfield microscopy, suggesting further invasion and deeper integration of the embryo inside the endometrial environment. At the centre of large trophoblast plaque, we could identify the cluster of OCT4+ cells orderly arranged around a central expanding pro-amniotic cavity (FIG. 3F). The SOX17+ hypoblast layer has covered the basal layer of the epiblast, while the yolk sac cavity further expanded compared to 10 d.p.f. Careful analysis of the implantation site revealed major structural transformation of the trophoblast, which has firmly anchored the embryo inside the stromal endometrial layer (FIG. 3G). Alongside single nucleated cells, which reminiscent of the proliferative stem cell population of the cytotrophoblast, large plate-like assemblies of multinucleated cells could be observed at the leading edge of the invading trophoblast plaque, corresponding to the syncytiotrophoblast. Of particular interest was the population of KRT7 expressing trophoblast cells which gave rise to an intricate network branching radially from the implantation centre (FIG. 3G). The columnar and ramification morphology would suggest an active role of this trophoblast population in mediating primary contact with the surrounding endometrial cells and the establishment of the early embryo-maternal interface required for placentation and long-term development of the conceptus till birth. Interesting the connection between embryo and maternal tissue was not only physical in nature, but appeared to involve a molecular crosstalk, as supported by the unexpected increase in glandular secretions (PEAP/Glycodelin) by the maternal endometrium upon implantation of the human embryos in culture.

### Example 4: Blastoids Undergo Adhesion and Implantation into the Endometrial Scaffold

Due to ethical, technical and legislative considerations, research using human embryos may be subject to restrictions in different countries around the world. Stem cell-based embryo models called blastoids (Kagawa et al. (2021) Nature, doi: 10.1038/s41586-021-04267-8; Yanagida et al. (2021) Cell Stem Cell 28:1016-1022.e4; and Yu et al. (2021) Nature 591:620-626) offer the opportunity to model aspects of the human blastocyst and therefore provide a valuable complementary system to study early development. Encouragingly, recent studies revealed the capacity of human blastoids to attach to the surface of monolayer endometrial cells (Kagawa et al. (2021); and Karvas et al. (2023) Cell Stem Cell 30:1148-1165.e7). These results indicate compatibility between blastoids and endometrial cells required for this first step of implantation in vitro. It is not known, however, whether blastoids can model all phases of human embryo implantation including deep invasion of the endometrial tissue.

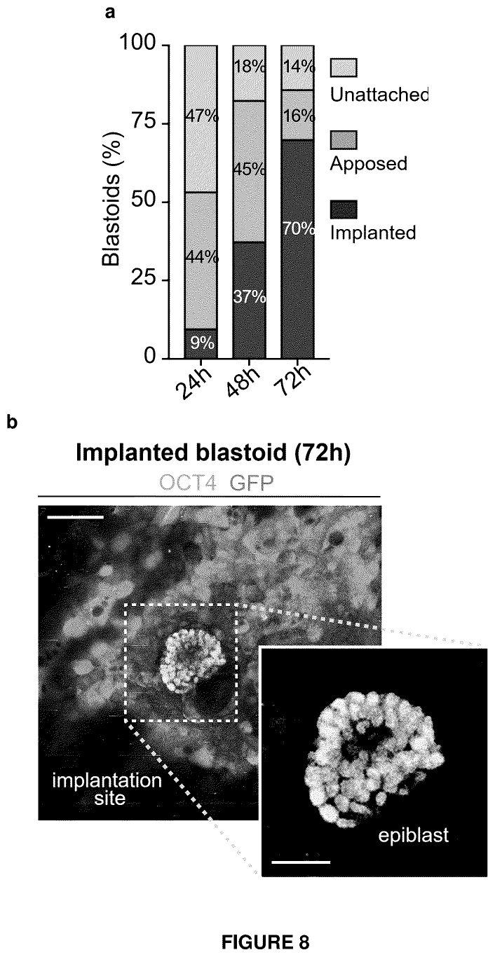

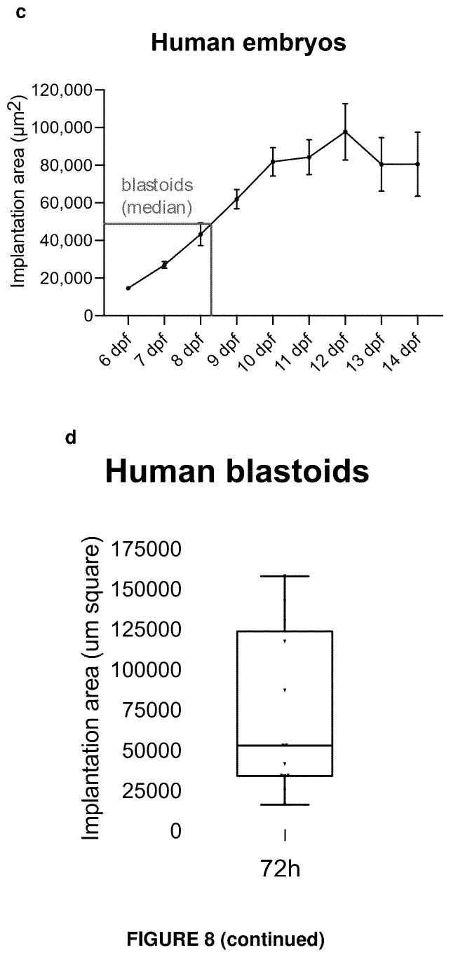

We generated blastoids from GFP-expressing human naïve embryonic stem cells and transferred the blastoids to the endometrial model to assess their ability to undergo implantation. After 24 hours, 44% of the blastoids initiated apposition to the surface of the luminal endometrium, retaining a clear expanded blastocoel cavity (FIG. 8A). After 48 hours, 37% of blastoids implanted by penetrating the endometrial epithelium and invaded into the endometrial stromal compartment (FIG. 8AI). This process peaked at 72 hours with 70% of blastoids achieving full implantation (FIGS. 8A and 8B). Although there was substantial variability, the median area of the blastoid implantation site at 72 hours was similar to the implantation area of human embryos between 8-9 d.p.f. (FIGS. 8C and 8D).

These results show that blastoids are able to reproduce the sequence of events of human embryo implantation inside the endometrial scaffold. Similar to human embryos, blastoids undergo initial apposition to the surface of the endometrium followed by adhesion and invasion into the deeper stromal layer. The high efficiency of blastoids undergoing successful implantation supports the co-culture of blastoids within the endometrial system as a complementary system to model human embryo implantation in vitro.

### Example 5: Characterisation of the Human Embryo Implantation Niche and Early Placentation

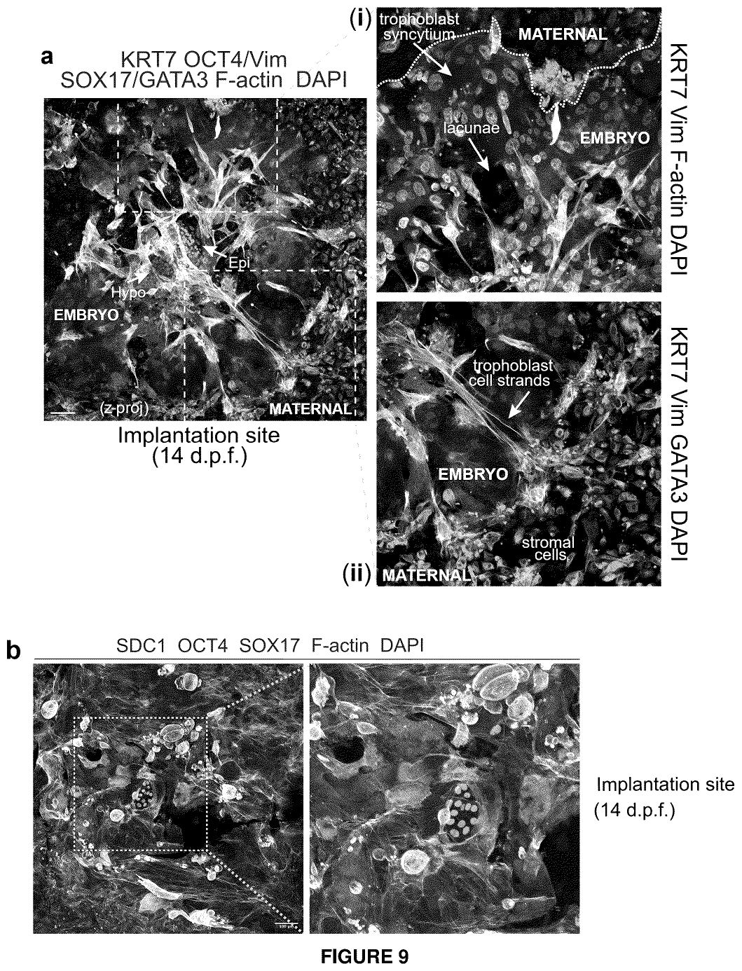

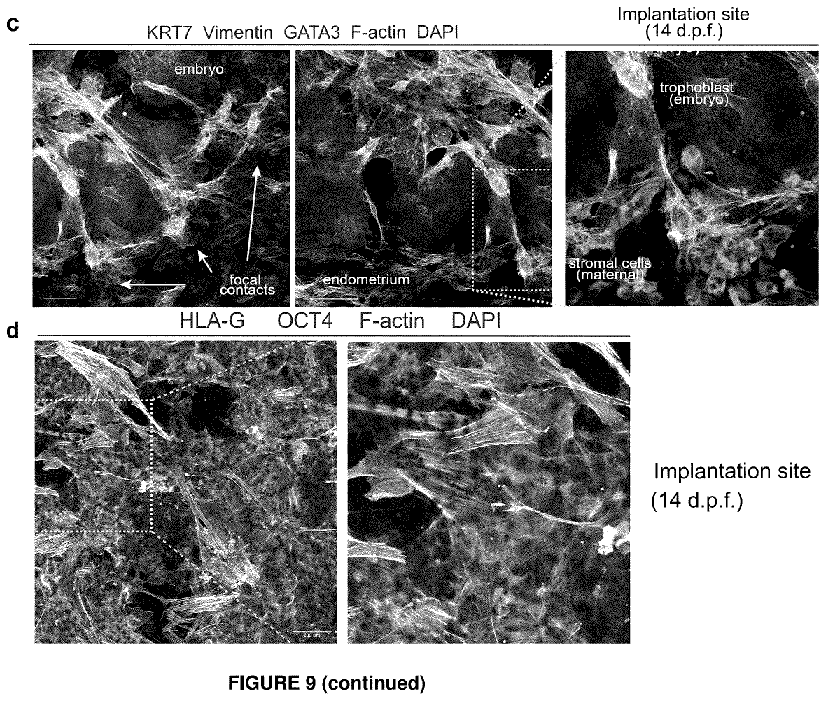

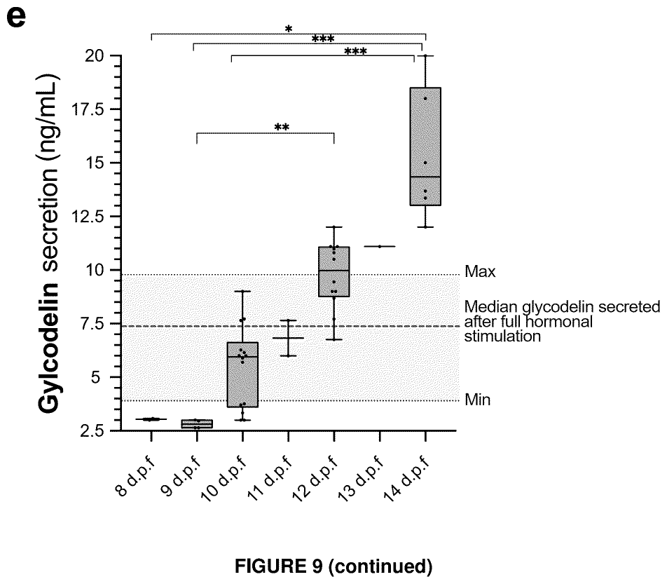

We next examined the implantation niche within the endometrial scaffolds at 14 d.p.f. of human embryo development. We observed the formation of large outgrowths from the embryo that were characterised by the extensive proliferation and differentiation of trophoblast cells (FIG. 9A). The trophoblast cells had fully invaded the endometrium and embedded within the stroma, as shown by VIM+ stromal cells surrounding the embryo (FIG. 9A). This observation is consistent with the deep interstitial invasiveness of the human conceptus. The trophoblastic outgrowths were characterised by numerous single-nucleated GATA3+ cytotrophoblast cells and by the formation of plate-like assemblies of multi-nucleated cells at the leading edge of the invading trophoblast cells (FIG. 9A (i), zoom). The presence of multi-nucleated cells that were SDC1+ confirms syncytial transformation of the trophoblast cells, which surrounded the entire conceptus (FIG. 9B). Among this syncytial mass, multiple lacunae were observed (FIG. 9A (i), zoom). Of particular interest was the appearance of uninuclear KRT7+ trophoblast cells that extended as strands through the syncytium to the margin of the implantation site where they spread laterally, forming what appeared to be the earliest stages of the cytotrophoblastic shell (FIG. 4A (ii), zoom). These strands represent the forerunners of the cytotrophoblast cell columns and characterise the transformation of the syncytial trabeculae into primary villi. These early villi were beginning to proliferate, branch and initiate focal anchorage contacts at multiple sites between the conceptus and the decidualised stromal cells (FIG. 9C). Many of the cells forming the trophoblast strands were HLA-G+, suggesting their differentiation into early extravillous trophoblast (FIG. 9D). This is the start of the embryo-maternal interface for the establishment of the early placenta, a process described in the historical anatomical sections from the Carnegie collection but has not been visualised before in living cultures.

Interestingly, the connection between embryo and maternal tissue also appeared to involve a paracrine interaction. This is supported by the increased levels of glycodelin secretion by the endometrium following implantation of embryos (FIG. 9E).

### Materials and Methods

### Participants and Biological Samples

Endometrial biopsies (n=15) were collected from 15 healthy women undergoing oocyte donation cycles. All participants met the criteria for oocyte donation program. All donors reported negative serologies for hepatitis B and C, human immunodeficiency virus (HIV), and syphilis. Donors were fertile women aged 18-31 years with a body mass index (BMI) of 19-25 kg/m², reporting normal menstrual cycles (21-35 days) and proven fertility. Correct uterine structure was confirmed in all donors by ultrasonographic and gynaecological examinations. Donors either carrying intra-uterine devices (IUD), reporting pathological conditions such as adenomyosis, endometriosis, endometritis, pelvic inflammatory disease, abnormal bleeding, other anatomic pathologies of the uterus or reporting pregnancy within three months prior of sample collection were excluded. Endometrial biopsies were collected with a pipette catheter (CCD Laboratories, France) from uterine fundus from oocyte donors on the day of oocyte retrieval. Inter-biopsy variability was limited because all donors underwent the same stimulation protocol prior oocyte retrieval. Endometrial samples were processed to isolate either endometrial stromal or epithelial fractions the same day of biopsy collection.

Isolation and Culture of Human Endometrial Stromal Cells (hEnSC) and Human Endometrial Epithelial Glandular Cells from Endometrial Biopsies and Passage.

Isolation of hEnSC was performed by gravity sedimentation. In brief, endometrial tissue collected the same day of isolation was mechanically fragmented into smaller pieces (1≤mm³) with scalpels. Minced fragments were enzymatically digested with collagenase IA (Sigma Aldrich, SCR103) diluted (1:10) in phenol red-free Dulbecco's Modified Eagle's Medium (DMEM, Sigma Aldrich, D4947) overnight at 4° C. After incubation period, tubes were vertically oriented for 10 minutes, allowing then the separation of epithelial and stromal compartments according to their different size by gravity sedimentation. Recovered supernatant containing hEnSCs were filtered (50-μm cell filters, Celltrics GmbH, Germany) and collected in new tubes. Three washes with DMEM (Sigma Aldrich, D4947) were performed to collect remaining hEnSCs. After each wash, superior phases were subsequently filtered and collected as described previously. After a centrifugation step (1000 g, 5 minutes) of collected supernatants, pellets of hEnSCs were obtained and resuspended in advanced DMEM/F12 medium (Gibco, Thermo Scientific, 12634010) supplemented with 10% of charcoal-stripped foetal bovine serum (FBS, Sigma Aldrich, F6765), 2 mM L-glutamine (Gibco, Thermo Scientific, 25030024) and 0.6% antibiotics (Penicillin-Streptomycin 10000 U/mL-10000 μg/mL; Gibco, Thermo Scientific, 15140122). Homogeneity of cultures was determined according to morphologic characteristics and verified by immunocytochemical localization of the stroma/epithelial marker vimentin and the immune cell/macrophage marker CD45 (Pérez-Deben et al., 2020). hEnSC were seeded and propagated in 25 cm² cell culture flasks (Nunc™ EasYFlask™ Cell Culture Flasks, Thermo Scientific, 156367). When cell confluence ranged between 80%-90% hEnSC were propagated (ratio 1:10). hEnSC included in this study were propagated up to passage 11. To passage hEnSC, culture medium was removed and 25 cm² flasks (Nunc™ EasYFlask™ Cell Culture Flasks, Thermo Scientific, 156367) were washed once with 2 mL of sterile Dulbecco's Phosphate Buffered Saline with calcium and magnesium (PBS, Sigma-Aldrich, D8662) and incubated with 2 mL of Accutase dissociation solution for 7 minutes at 37° C. Enzymatic action was stopped by diluting cell suspension in Accutase in 8 mL of hEnSC medium supplemented with 10% of charcoal stripped FBS (Sigma Aldrich, F6765), 2 mM L-glutamine (Gibco, Thermo Scientific, 25030024) and 0.6% antibiotics (Penicillin-Streptomycin 10000 U/mL-10000 μg/mL; Gibco, Thermo Scientific, 15140122). After centrifugation (600 g, 6 minutes), supernatant was discarded, and pellet was resuspended with 1 mL fresh hEnSC medium supplemented with 10% of charcoal-stripped FBS (Sigma Aldrich, F6765), 2 mM L-glutamine (Gibco, Thermo Scientific, 25030024) and 0.6% antibiotics (Penicillin-Streptomycin 10000 U/mL-10000 μg/mL; Gibco, Thermo Scientific, 15140122). Cell dilution (1:10) was seeded in a new 25 cm² flask (Nunc™ EasYFlask™ Cell Culture Flasks, Thermo Scientific, 156367) and cultured with hEnSC medium supplemented with 10% of charcoal stripped FBS (Sigma Aldrich, F6765), 2 mM L-glutamine (Gibco, Thermo Scientific, 25030024) and 0.6% antibiotics (Penicillin-Streptomycin 10000 U/mL-10000 μg/mL; Gibco, Thermo Scientific, 15140122).

In order to isolate human endometrial epithelial glandular fractions, after mechanical disaggregation of endometrial tissue, fragments were enzymatically digested with a mixture of dispase II (1.25 U/mL, Sigma-Aldrich, D4693) —collagenase V (0.4 mg/mL, Sigma-Aldrich, C-9263) diluted in RPMI 1640 medium (Thermo Fisher Scientific, 21875-034) supplemented with 10% fetal calf serum (FCS, Biosera, FB-1001) for 30 minutes at 37° C. in shaking. Supernatant was filtered (100 μm cell sieves, Sigma-Aldrich, 431752) and glands were retained. Endometrial epithelial glands were pelleted by centrifugation (600 g, 6 minutes) and resuspended in ice-cold Geltrex (Geltrex™ LDEV-Free, hESC-Qualified, Reduced Growth Factor Basement Membrane Matrix; Gibco, Thermo Scientific, USA, A1413301). Endometrial epithelial gland fragments resuspended in Geltrex were seeded in 25 μL domes in 48-well plate (Corning Costar, 3548) and were covered with 250 μL of organoid medium modified from Turco et al., 2017. In brief, Advanced DMEM/F12, Gibco, 12634-010) was supplemented with B27 (Gibco, 12587-010), N2 (Gibco, 17502-048), 2 mM L-glutamine (Gibco, Thermo Scientific, 25030024), 1.25 mM N-Acetyl-L-cysteine (NAC, Sigma, A9165), 10 mM nicotinamide (Sigma, N0636), recombinant human Rspondin-1 (80 ng/mL, Peprotech, 120-38), recombinant human fibroblast growth factor 10 (FGF-10 100 ng/ml, Peprotech, 100-26), recombinant human noggin (100 ng/ml, Peprotech, 120-10c), recombinant human epidermal growth factor (EGF, 50 ng/mL, Peprotech, AF-100-15), recombinant human hepatocyte growth factor (HGF, 50 ng/ml, Peprotech, 100-39), ALK-4, -5, -7 inhibitor (A83, 500 nM, Tocris, 2939) and 0.2% antibiotics (Penicillin-Streptomycin 10000 U/mL-10000 μg/mL; Gibco, Thermo Scientific, 15140122).

Endometrial epithelial organoid medium was changed each 2 days and organoids domes were passaged (ratio 1:3) each 7-10 days depending on the dome's confluence. Organoid passage was performed by manual pipetting. Briefly, Geltrex domes containing the endometrial epithelial organoids were disaggregated. Wells were washed once with advanced DMEM/F12 (Gibco, Thermo Fisher, 12634010). Suspension containing fragmented Geltrex domes was centrifuged (600 g, 6 minutes) and upper phase was discarded. Remaining Geltrex phase and pellet were dissociated by manual pipetting (×400 times) and suspension was centrifuged again (600 g, 6 minutes). All supernatant was discarded and the pellet containing endometrial epithelial fragments was dissociated using by manual pipetting (×100 times). After centrifugation (600 g, 6 minutes), the pellet of endometrial epithelial fragments was resuspended in ice-cold Geltrex and fresh Geltrex domes (25 μL) were seeded in 48-well plate (Corning Costar, 3548) and were covered with 250 μL of organoid medium.

### Assembling Human Endometrial Stromal and Epithelial Components in a Three-Dimensional Culture System.

The three-dimensional in vitro model recreating human endometrium based on a transwell culture system (Greiner Bio-One ThinCert™ Tissue Culture Inserts 662630) was designed with the aim of satisfying the need to obtain a physiological matrix that allows the study of embryo development in the peri-implantation context. The in vitro culture system proposed allows communication between the lower and upper compartments through a porous membrane; thus, exchange between two compartments is facilitated through cell layers while culture media from upper and lower compartments are kept isolated. Consequently, this fact prevents the mixing of both media; thus, permitting the differential supply of nutrients, factors, and hormones to the upper and lower compartments. The culture system included a stromal compartment within cells forming a three-dimensional network embedded in a tuneable hydrogel containing main extracellular matrix proteins found in human endometrium. Epithelial compartment, including both luminal and glandular components, was then seeded on top to recapitulate epithelial barrier above stromal matrix. Therefore, the transwell-based in vitro culture system enabled the stromal to nourish from the medium in the lower compartment while medium in the top compartment fulfilled the different nutritional demand of epithelial component and concentrated the majority of secreted products as in the endometrial lumen.

Prior to incorporate hEnSC in the hydrogel enriched with extracellular protein matrix, the chemically defined hydrogel backbone was obtained using a commercial kit (TrueGel3D Hydrogel Kit, Sigma-Aldrich, TRUE7). Dextran polymers and buffer solution provided by the kit were mixed with most abundant extracellular matrix proteins in human endometrium. Extracellular matrix proteins were incorporated in a specific ratio corresponding to their relative abundance in endometrial tissue. Accordingly, 45% of collagen III (Rockland immunochemicals, 009-001-105), 35% of collagen I (Rockland immunochemicals, 009-001-103), 10% of collagen VI and 10% of fibronectin were incubated in ice for 1 hour at 4° C. in presence of dextran polymers and buffer solution. As extracellular matrix proteins provided an acid environment, after incubation period, pH of the solution was neutralised by adding 29.16% of hEnSC medium supplemented with 10% charcoal stripped FBS (Sigma Aldrich, F6765), 25 mM HEPES (Gibco, Thermo Scientific, 15630080) and 76.67 mM sodium bicarbonate. Alongside the neutralisation period at room temperature, concentration of sodium bicarbonate could be increased up to 84.97% to facilitate the acquisition of pH=7. After neutralization, 100.000 hEnSC per transwell passaged as described above were incorporated into the dextran solution enriched with extracellular matrix proteins together with the cyclodextrin crosslinkers (10% of final hydrogel volume). A total of 30 μL of hydrogel containing hEnSC and enriched with extracellular matrix proteins was seeded in each transwell. Hydrogel was allowed to polymerize during 25 minutes at 37° C. 7% CO2, being the first 7 minutes incubated as hanging drops to avoid meniscus formation. After full polymerization the final stiffness of the hydrogel was tuned to 250 Pa in order to recreate the in vivo stiffness of human endometrium. Hydrogel with hEnSC embedded were cultured in vitro in the transwell system with hEnSC medium supplemented with 10% charcoal stripped FBS (Sigma Aldrich, F6765) in upper and lower compartments to permit hEnSC proliferate and elongate. hEnSC embedded in the hydrogel were in vitro cultured for 11-12 days in hEnSC medium supplemented with 10% charcoal-stripped FBS (Sigma Aldrich, F6765), changing the medium each 4 days.

Once hEnSC proliferated and exhibited elongated fibroblast-like morphology being spatially arranged alongside hydrogel volume, endometrial epithelial organoids were fragmented and seeded on top of the stromal compartment. Endometrial epithelial organoids were passaged and dissociated by manual pipetting as described previously. Endometrial epithelial fragments were resuspended in organoid medium modified from Turco et al., 2017 supplemented with 2% charcoal stripped FBS (Sigma Aldrich, F6765) and cultured in vitro in the upper transwell compartment. Medium change of the top compartment was changed each 2 days, while hEnSC media supplemented with 10% charcoal stripped FBS (Sigma Aldrich, F6765) was maintained in the lower compartment. Epithelial and stromal components were kept proliferating during 4 to 6 days to allow epithelial fragments to outgrowth and coat most of the surface of the transwell.

### Hormone Stimulation of Human Endometrial Scaffolds

Hormone stimulation scheme aimed to recapitulate the mid-secretory phase of human endometrial cycle when receptive phenotype optimal for embryo implantation, known as window of implantation, is acquired. For this purpose, β-oestradiol (E2) stimulation was started in upper and lower compartments for 4 days to simulate the proliferative phase of human endometrial cycle. A total volume of 300 μL of organoid media supplemented by 2% charcoal stripped FBS (Sigma Aldrich, F6765) and 10 nM of β-oestradiol (E2, Sigma E4389) was added to the top compartment of the transwell culture system. A total volume of 1 mL of hEnSC medium supplemented by 2% charcoal stripped FBS (Sigma Aldrich, F6765) and 10 nM of β-oestradiol (E2, Sigma E4389) was added to the lower compartment. Media were changed each 2 days. After 4 days, upper and lower compartments were stimulated with full hormone mixture containing E2, progesterone (P4) and 8-bromoadenosine 3′,5′-cyclic monophosphate (CAMP) for 8 days. A total volume of 300 μL of organoid media supplemented by 2% charcoal stripped FBS (Sigma Aldrich, F6765), 10 nM of E2 (Sigma E4389), 1 μM of P4 (Sigma P7556) and 1 μM of cAMP (Sigma, B7880) was added to the top compartment of the transwell culture system. A total volume of 1 mL of hEnSC medium supplemented by 2% charcoal stripped FBS (Sigma Aldrich, F6765) and nM of E2 (Sigma E4389), 1 μM of P4 (Sigma P7556) and 1 M of cAMP (Sigma, B7880) was added to the lower compartment. Media were changed each 2 days.

### Acquisition Endometrial Receptivity in Endometrial Scaffolds

In vivo human endometrium is finely regulated by hormones. Ultimately, alongside the menstrual cycle all endometrial cell types become responsive to hormones acquiring a functional secretory phenotype optimal for embryo implantation. Accordingly, for the endometrium to become receptive is necessary that the cells composing this tissue undergo profound morphological and biochemical changes that make these cells competent to support embryo development. A major hallmark of human endometrial receptivity is decidualization of human endometrial stromal cells (hEnSC). Hormonal stimulus provided by ovarian hormones E2 and P4 prime hEnSC to go through a mesenchymoepithelial differentiation evinced by a morphological change from elongated fibroblast-like morphology to a cuboidal shape and to acquire a secretory phenotype (B. Gellersen et al., 2007). Among the main products secreted by decidual hEnSCs, prolactin (PRL) and insulin-like growth factor binding protein 1 (IGFBP-1) are two proteins that have been widely used as phenotypic markers of decidualization (B. B. Gellersen & Brosens, 2014). Similarly, hormonal action of E2 and P4 also drive morphological changes in endometrial epithelium, often characterized by the appearance of pinopodes which are globular protrusion-like cell structures appearing on the apical surface of the luminal epithelium during the mid-secretory phase. Appearance of pinopodes resulting from the fusion of microvilli is considered a marker of endometrial receptivity as pinopodes appearance coincides with the window of implantation and they may play an important role in facilitating contact between the embryo and the endometrium (Nikas & Makrigiannakis, 2003). Further to morphological changes, human endometrial epithelial cells (hEnEC) also acquire a secretory phenotype in response to hormonal stimulation. One of the main products secreted as a consequence of the acquisition of the secretory phenotype is glycodelin (PAEP). Glycodelin is one of the main components of the secretions produced by the endometrial glands, known as “uterine milk” (Burton et al., 2020).

### Quantifying the Secretions in Hormone-Stimulated Endometrial Scaffolds.

As hormone-responsiveness capability and endometrial receptivity are acquired progressively along the menstrual cycle, quantification of secreted products and morphometric analyses were evaluated before stimulation (unstimulated phase), proliferative phase when only E2 stimulation is considered and in two different time-points of secretory phase (after 4 and 8 days of inducing stimulation with E2, P4 and CAMP). The set of products secreted by the functional hormone-responsive endometrium which contribute to provide the necessary histotrophic nutrition for the embryo in the peri-implantational stages was assessed by enzyme-linked immunosorbent assays (ELISA). Secreted levels of PRL (Human Prolactin ELISA Kit, R&D Systems, DPRL00), IGFBP-1 (ELISA Kit for Insulin Like Growth Factor Binding Protein 1, Clone Cloud Corp, SEA052Hu) and PAEP (Human PAEP ELISA Kit, Abcam, ab275904) were quantified in culture media supernatants from upper and lower compartments of the transwell-based culture system. In all cases, the above-mentioned timepoints along hormonal stimulation regimen were considered. Two technical replicates were included. Secreted concentration of the proteins tested was obtained after obtaining the mean value of the technical replicates and normalizing the optical density measurements of each sample either to organoid medium supplemented with 2% charcoal stripped FBS (Sigma Aldrich, F6765) or hEnSC medium. Finally, standard curve run for each protein tested was used to convert optical density measurements into concentration values. Therefore, all optical density measurements and following calculated concentrations correspond to secreted levels of different proteins tested by the cells in the system.

### Cell Segmentation and Cell Morphometric Analyses of Stromal and Epithelial Cells in Hormone-Stimulated Endometrial Scaffolds.

Morphometric analyses were performed on stromal and epithelial compartments on proliferative phase (after 4 days of E2 stimulation) and secretory phase (after 8 days of stimulation with E2, P4 and cAMP).

Cell segmentation and cell morphometric analyses were performed from confocal microscopy images of stromal and epithelial compartment. Vimentin and cytokeratin-7 were used as stromal and epithelial markers, respectively, to determine stromal and epithelial compartment. Phalloidin signal was used to perform cell segmentation of stromal and epithelial compartments. Cell segmentation was done in an intermediate field of the whole Z-stack, choosing a relatively flat area to maximize the accuracy of the segmentation using Fiji (https://imagej.net/software/fiji/).

Epithelial cells were segmented manually using polygon selection tool from Fiji. Cell contour was drawn with multiple points to accurately fit perimeter of each cell segmented. Fit spline option (“Edit”>“Selection”>“Fit spline”) was used in all cases to smooth cell contour and maximize the match of manual cell segmentation with cell perimeter. Each Region Of Interest (ROI) corresponding to each epithelial cell segmented was added to ROI manager (“Analyse”>“Tools” >“ROI manager”).

Once acquired all ROI, the following measurements were set (“Analyse”>“Set Measurements”): area (μm²), perimeter (μm), and shape descriptors roundness, circularity, aspect ratio, and solidity. Roundness ((4·area)/(π·major axis²)) ranges from 0 to 1 indicating elongated or circle-like shape. Circularity (4π·area/perimeter²) ranges from 0 to 1 and correlates with shape roughness. Aspect ratio is defined as the ratio between major and minor axis of the best fit ellipse. Solidity, defined as a ratio between area and convex area, ranges from 0 to 1, describing a concave or convex shape.

Stromal length (μm) and width (μm) were acquired for each cell using the line tool from Fiji. Each Region Of Interest (ROI) corresponding to each epithelial cell segmented was added to ROI manager (“Analyse”>“Tools”>“ROI manager”). In this case, only line measurements (in μm) corresponding to length and width of each stromal cell were recorded to calculate length/width ratio. Higher values of length/width ratio indicate elongated fibroblast-like morphology while length/width ratio closer to 1 indicates cuboidal morphology characteristic of decidual hEnSC.

### Human Embryo Culture in Hormone-Stimulated Endometrial Scaffolds

The use of human blastocysts was approved by the National Commission of Human Assisted Reproduction (CNRHA; reference code 32544/72212). Supernumerary 5 d.p.f. blastocysts (n=103) from in vitro fertilization cycles donated for research purposes were included in this study after written and signed informed consent was obtained from the progenitors.

Blastocyst thawing was performed according Kitazato protocol (Kitazato Corporation) for embryo thawing. Blastocysts were handled using a micropipetter (Origio, MXL3-STR-CGR) and micropipetter flexible tips (Origio, MXL3-275). Open-pulled straws where 5 d.p.f. blastocysts were vitrified were transferred directly from liquid nitrogen (LN) and immersed in 1 mL pre-warmed thawing solution (TS, Kitazato, Hunter Scientific, VT802-2). To facilitate the immersion of the open-pulled straw in TS, the solution was poured in a 58 cm² petri dish (Thermo Scientific, 263991). After 1 minute immersed in TS, 5 d.p.f. blastocysts were completely release from the open-pulled straw and transferred to a ReploPlate to continue thawing process (ReproPlates plate, Kitazato, Hunter Scientific, 83020). 5 d.p.f. blastocysts were transferred consecutively into 300 μL of Dilution Solution (DS, Kitazato, Hunter Scientific, VT802-2) for 3 minutes, 300 μL of Washing Step 1 Solution (WS1, Kitazato, Hunter Scientific, VT802-2) for 5 minutes and 300 μL Washing Step 2 Solution (WS2, Kitazato, Hunter Scientific, VT802-2) for 1 minute. During this process, 5 d.p.f. blastocysts were carefully handled minimizing the volume of media transferred in each step.

Following the second washing step, 5 d.p.f. blastocysts were transferred into drops of pre-equilibrated medium (37° C., 20% O₂ and 5% CO2) for culture of pre-implantation embryos (SAGE 1 step, Origio, REF67010060A) covered by mineral oil (Ovoil, Vitrolife, Life Technologies, 10029). 5 d.p.f. blastocysts were washed twice, then transferred to individual drop of culture media and were incubated (37° C., 20% O₂ and 5% CO2) for 5 hours to allow recovery after thawing.

After 5 h incubation in SAGE 1-Step (Origio, REF67010060A), 5 d.p.f. expanded blastocysts were transferred into hormone-stimulated scaffolds. Prior transferring 5 d.p.f. blastocysts into endometrial scaffolds, the top compartments of the transwell culture system were pre-equilibrated (37° C., 20% O₂ and 5% CO2 for a minimum of 3 hours) with in-house formulated human embryo medium (HEM-1). HEM-1 contains a basal medium (Advanced DMEM/F12, Gibco, 12634-010) supplemented with B27 (Gibco, 12587-010), N2 supplement (Gibco, 17502-048), 2 mM L-glutamine, 1.25 mM N-Acetyl-L-cysteine (NAC, Sigma, A9165), 10 mM nicotinamide (Sigma, N0636), 2% charcoal stripped FBS (Sigma Aldrich, F6765), 0.5 mg/ml of hyaluronan (Abcam, Ab143634), growth factors (50 ng/ml of insulin-like growth factor 1 IGF-1, Cell Guidance Systems GFH34AF and 50 ng/ml of epidermal growth factor EGF, Peprotech, AF-100-15), hormones released from maternal (10 nM of E2, Sigma E4389; 1 μM of P4, Sigma P7556 and prolactin) and a minimal background of hormones secreted from embryo end (1 μg/mL of human Chorionic Gonadotropin hCG and 20 ng/ml of human Placental Lactogen hPL). Lower compartments of the transwell culture system which are only in contact with hEnSC were filled with 1 mL of hEnSC medium supplemented by 2% charcoal stripped FBS (Sigma Aldrich, F6765), 10 nM of E2 (Sigma E4389) and 1 μM of P4 (Sigma P7556). Media from lower compartments were changed each 2 days. As explained above, the transwell culture system chosen allows only the communication within different cell layers but the porous membrane prevent media from the upper and lower compartments to be mixed. Hence, only embryos and epithelial component were in contact with HEM-1, while stromal component in the base of the transwell benefited from the culture medium below which contained the nutrients needed for stromal growth and hormonal supply. Embryos were cultured in HEM-1 (350 μL) from 5 d.p.f. to 9 d.p.f. Half volume of HEM-1 medium was replaced daily with 200 μL of fresh HEM-1. Replacing only half of the volume allowed paracrine signaling while new supply of fresh nutrients was provided to enhance embryo survival. Upon attachment 10 μM of ROCK inhibitor (Y-27632) was added to HEM-1. Embryo attachment was defined as the blastocoel collapse and loss of expanded morphology.

As nutrient requirements of embryos increased from 9 d.p.f. onwards, a second in-house designed human embryo media (HEM-2) was introduced, replacing HEM-1. HEM-2 contained all compounds present in HEM-1, charcoal stripped FBS concentration (Sigma Aldrich, F6765) was increased from 2% to 5%, 7.5 mM glucose (Sigma, G8644), Insulin-Transferrin-Selenium-Ethanolamine (Gibco, 51500-056), essential and non-essential amino acids (Gibco 11130-036 and Gibco 11140-035) were supplemented. The extra supply of nutrients contributed mainly to provide nutrients to trophoblast as it started to proliferate vigorously. Embryos were cultured in HEM-2 (450 μL) from 9 d.p.f. to 12 d.p.f. Half volume of HEM-2 medium was replaced daily with 250 μL of fresh HEM-2. Lower compartments of the transwell culture system which are only in contact with hEnSC were kept in culture with the same medium as detailed above, being replaced by fresh medium each 2 days.

The last two days of human embryo culture, embryos from 12 d.p.f to 14 d.p.f. were cultured in a third in-house designed medium (HEM-3) which contained all components of HEM-2, but charcoal stripped FBS concentration (Sigma Aldrich, F6765) was increased up to 10%.

### Morphometric Analysis and Cell Lineage Quantification in Human Embryo During In Vitro Culture Up to 14 d.p.f in Hormone-Stimulated Endometrial Scaffolds

Bright field pictures of each embryo during in vitro culture up to 14 d.p.f in endometrial scaffolds were recorded using an inverted microscope (Axio Zeiss Vert A.1) coupled to temperature-controlled plate.

Human embryo segmentation and morphometric analyses were performed using Fiji (https://imagej.net/software/fiji/). Prior to start segmentation and morphometric analyses, scale was set manually (“Analyse”>“Set scale”) by introducing the pixels and μm of each objective used to obtain a pixel/μm ratio. Human embryos were then segmented manually using polygon selection tool from Fiji. Cell contour was drawn with multiple points to accurately fit perimeter of each cell segmented. Fit spline option (“Edit”>“Selection”>“Fit spline”) was used in all cases to smooth cell contour and maximize the match of manual cell segmentation with human embryo perimeter. Each Region Of Interest (ROI) corresponding to each embryo segmented was added to ROI manager (“Analyse”>“Tools”>“ROI manager”). Once acquired all ROI, the following measurements were set (“Analyse”>“Set Measurements”): area (μm²), perimeter (μm), major and minor axis (μm) which corresponded to long and short diameter and shape descriptors roundness, circularity, aspect ratio, and solidity defined above.

Cell lineage quantification were performed from confocal microscopy images of 10 d.p.f. (n=2) and 14 d.p.f (n=2) implanted human embryos in hormone stimulated endometrial scaffolds. Oct4 (mouse 1:100, Santa Cruz Biotechnology, sc5279), Sox 17 (goat 1:100, R&D, AF1924) and Gata3 (rabbit 1:200, Abcam, ab199428) antibodies were used for immunolocalization and quantification of epiblast, hypoblast, and trophoblast lineages respectively. Images were acquired setting bidirectional imaging, resolution at 1024×1024 pixels, 600 speed, frame averaging: 1, format 8-bit. Number of cells per lineage were quantified alongside the whole Z-stack using dot tool of Fiji (https://imagej.net/software/fiji/) to track the cells counted.

Embryo-Endometrial Secretory Markers During In Vitro Human Embryo Development Up to 14 d.p.f. In Hormone-Stimulated Endometrial Scaffolds

The first confirmation of biochemical pregnancies is the detection of human chorionic gonadotropin (hCG) in maternal peripheral blood and or urine. hCG is an essential hormone secreted by the trophoblast in its early development which exerts different functions related to supporting embryo development and maintenance of pregnancy (D'hauterive et al., 2022). Hence, secreted hCG levels by human embryos on peri and post-implantation stages were quantified and tracked during in vitro culture in hormone-stimulated endometrial scaffolds. Furthermore, aiming to track the changes in endometrial secretions induced by the presence of the embryo, PAEP secreted levels were also quantified in hormone-stimulated scaffolds cultured in vitro with human embryos using the same ELISA kit (Human PAEP ELISA Kit, Abcam, ab275904) detailed above. hCG concentration released by human embryos was quantified by ELISA (Free beta-Human Chorionic Gonadotropin (b-hCG) ELISA kit. Origene. CAT #: EA100983). PAEP and b-hCG secreted levels were measured in culture media supernatants from upper compartments of the transwell-based culture system on different days of embryo development (from 6 d.p.f to 14 d.p.f). In this case, as the volume of culture media collected was limited, no technical replicates were included. Secreted concentration of PAEP and b-hCG were obtained after normalizing the optical density measurements of each sample to HEM2 (incorporated as a blank). HEM-2 was used as a blank because the majority of the samples were included in this media. The composition between HEM-1 and HEM-3 respectively with HEM-2 is still similar and all the 3 media used for embryo culture have the same concentration of b-hCG incorporated in the media. Therefore, all optical density measurements and following calculated concentrations correspond to secreted levels of PAEP and b-hCG by embryos in the system. Finally, standard curves run were used to convert optical density measurements into concentration values.

Propagation of Human Endometrial Stromal Cells (hEnSC)

| | Company and | Final |
|---|---|---|
| Reagent | catalogue/reference number | concentration |
| Advanced DMEM/F-12 | Thermo Scientific. Gibco. | 1X |
| | Catalog number: 12634010 | |
| Fetal Bovine Serum | Sigma-Aldrich. | 10% |
| (charcoal-stripped) | Reference number: F6765 | |
| L-glutamine | Thermo Scientific. Gibco. | 2 mM |
| | Catalog number: 25030024 | |
| Penicillin/Streptomycin | Thermo Scientific. Gibco. | Pen: 60 u/mL |
| | Cat#: 15140122 | Strep: 60 ug/mL |

### Propagation of Human Endometrial Epithelial Organoids

| | Company and | Final | |
|---|---|---|---|
| Reagent | catalogue/reference number | concentration | |
| Advanced DMEM/F-12 | Thermo Scientific. Gibco. | 1X | |
| | Catalog number: 12634010 | | |
| N2 supplement | Thermo Scientific. Gibco. | 1X | |
| | Catalog number: 17502048 | | |
| B2 supplement minus | Thermo Scientific. Gibco. | 1X | |
| vitamin A | Catalog number: 12587010 | | |
| Nicotinamide | Sigma | 10 | mM |
| | Reference: N0636 | | |
| N-Acetyl-L-cysteine | Sigma | 1.25 | mM |
| | Reference number: A9165-5G | | |
| L-glutamine | Thermo Scientific. Gibco. | 2 | mM |
| | Catalog number: 25030024 | | |
| Penicillin/Streptomycin | Thermo Scientific. Gibco. | 20 | U/mL |
| | Cat#: 15140122 | | |
| Recombinant human | Peprotech | 80 | ng/mL |
| Rspondin-1 | Reference number: 120-38 | | |
| Recombinant human | Peprotech | 100 | ng/mL |
| FGF-10 | Reference number: 100-26 | | |
| Recombinant human | Peprotech | 100 | ng/mL |
| Noggin | Reference number: 120-10c | | |
| Recombinant human | Peprotech | 50 | ng/mL |
| EGF | Reference number: AF-100-15 | | |
| Recombinant human | Peprotech | 50 | ng/mL |
| HGF | Reference number: 100-39 | | |
| A83 (ALK-4,-5,-7 | Tocris | 500 | nM |
| inhibitor) | Reference number: 2939 | | |

Human Embryo Medium 1 (HEM-1). In-House Designed Human Embryo Culture Medium to Promote Human Embryo Development and Survival from 5 d.p.f. To 9 d.p.f.

| | Company and | Final | |
|---|---|---|---|
| Reagent | catalogue/reference number | concentration | |
| Advanced DMEM/F-12 | Thermo Scientific. Gibco. | 1X | |
| | Catalog number: 12634010 | | |
| N2 supplement | Thermo Scientific. Gibco. | 1X | |
| | Catalog number: 17502048 | | |
| B2 supplement minus | Thermo Scientific. Gibco. | 1X | |
| vitamin A | Catalog number: 12587010 | | |
| Nicotinamide | Sigma | 10 | mM |
| | Reference: N0636 | | |
| N-Acetyl-L-cysteine | Sigma | 1.25 | mM |
| | Reference number: A9165-5G | | |
| L-glutamine | Thermo Scientific. Gibco. | 2 | mM |
| | Catalog number: 25030024 | | |
| Penicillin/Streptomycin | Thermo Scientific. | 20 | U/mL |
| | Gibco. | | |
| | Cat#: 15140122 | | |
| Charcoal stripped foetal | Sigma Aldrich, | 2% | |
| bovine serum (FBS) | Reference number: F6765 | | |
| Human Serum Albumin | Vitrolife REF 10064 | 5% | |
| (HSA) | | | |
| Hyaluronan | Abcam | 0.5 | mg/mL |
| | Catalog number: Ab143634 | | |
| β-oestradiol (E2) | Sigma Aldrich | 10 | nM |
| | Reference number: E4389 | | |
| Progesterone (P4) | Sigma Aldrich | 1 | μM |
| | Reference number: P7556 | | |
| Human prolactin | Peprotech | 20 | ng/mL |
| (PRL) | Reference number: 100-07 | | |
| Human chorionic | Alpha Diagnostic Intl | 1 | μg/mL |
| gonadotropin (hCG) | Catalog number: RP-1484 | | |
| Human placental | R&D | 20 | ng/mL |
| lactogen (hPL) | Catalog number: 5757-PL/CF | | |
| ROCK inhibitor | Y-27632 | 10 | uM |
| (*) only added upon | | | |
| embryo attachment | | | |

Human Embryo Medium 2 (HEM-2). In-House Designed Human Embryo Culture Medium to Promote Human Embryo Development and Survival from 9 d.p.f. To 12 d.p.f.

| | Company and | Final | |
|---|---|---|---|
| Reagent | catalogue/reference number | concentration | |
| Advanced DMEM/F-12 | Thermo Scientific. Gibco. | 1X | |
| | Catalog number: 12634010 | | |
| N2 supplement | Thermo Scientific. Gibco. | 1X | |
| | Catalog number: 17502048 | | |
| B2 supplement minus | Thermo Scientific. Gibco. | 1X | |
| vitamin A | Catalog number: 12587010 | | |
| Nicotinamide | Sigma. | 10 | mM |
| | Reference: N0636 | | |
| N-Acetyl-L-cysteine | Sigma. | 1.25 | mM |
| | Reference number: A9165-5G | | |
| L-glutamine | Thermo Scientific. Gibco. | 2 | mM |
| | Catalog number: 25030024 | | |
| Penicillin/Streptomycin | Thermo Scientific. Gibco. | 20 | U/mL |
| | Cat#: 15140122 | | |
| Charcoal stripped foetal | Sigma Aldrich. | 5% | |
| bovine serum (FBS) | Reference number: F6765 | | |
| Human Serum Albumin | Vitrolife REF 10064 | 5% | |
| (HSA) | | | |
| Hyaluronan | Abcam. | 0.5 | mg/mL |
| | Catalog number: Ab143634 | | |
| β-oestradiol (E2) | Sigma Aldrich. | 10 | nM |
| | Reference number: E4389 | | |
| Progesterone (P4) | Sigma Aldrich. | 1 | μM |
| | Reference number: P7556 | | |
| Human prolactin | Peprotech. | 20 | ng/mL |
| (PRL) | Reference number: 100-07 | | |
| Human chorionic | Alpha Diagnostic Intl. | 1 | μg/mL |
| gonadotropin (hCG) | Catalog number: RP-1484 | | |
| Human placental | R&D. | 20 | ng/mL |
| lactogen (hPL) | Catalog number: 5757-PL/CF | | |
| ROCK inhibitor | Y-27632 | 10 | uM |
| (*) only added upon | | | |
| embryo attachment | | | |
| Glucose | Sigma Aldrich. | 7.5 | mM |
| | Reference number: G8644 | | |

| | | |
|---|---|---|
| Insulin-Transferrin- | Thermo Scientific. Gibco. | 1X |
| Selenium-Ethanolamin | Catalog number: 51500-056 | |
| (ITS-X) | | |
| Non-essential amino | Thermo Scientific. Gibco. | 1X |
| acids | Catalog number: 11140-035 | |
| Essential amino acids | Thermo Scientific. Gibco. | 1X |
| | Catalog number: 11130-036 | |

Human Embryo Medium 3 (HEM-3). In-House Designed Human Embryo Culture Medium to Promote Human Embryo Development and Survival from 12 d.p.f. To 14 d.p.f.

| | Company and | Final | |
|---|---|---|---|
| Reagent | catalogue/reference number | concentration | |
| Advanced DMEM/F-12 | Thermo Scientific. Gibco. | 1X | |
| | Catalog number: 12634010 | | |
| N2 supplement | Thermo Scientific. Gibco. | 1X | |
| | Catalog number: 17502048 | | |
| B2 supplement minus | Thermo Scientific. Gibco. | 1X | |
| vitamin A | Catalog number: 12587010 | | |
| Nicotinamide | Sigma. | 10 | mM |
| | Reference: N0636 | | |
| N-Acetyl-L-cysteine | Sigma. | 1.25 | mM |
| | Reference number: A9165-5G | | |
| L-glutamine | Thermo Scientific. Gibco. | 2 | mM |
| | Catalog number: 25030024 | | |
| Penicillin/Streptomycin | Thermo Scientific. Gibco. | 20 | U/mL |
| | Cat#: 15140122 | | |
| Charcoal stripped foetal | Sigma Aldrich. | 10% | |
| bovine serum (FBS) | Reference number: F6765 | | |
| Human Serum Albumin | Vitrolife REF 10064 | 5% | |
| (HSA) | | | |
| Hyaluronan | Abcam. | 0.5 | mg/mL |
| | Catalog number: Ab143634 | | |
| β-oestradiol (E2) | Sigma Aldrich. | 10 | nM |
| | Reference number: E4389 | | |
| Progesterone (P4) | Sigma Aldrich. | 1 | μM |
| | Reference number: P7556 | | |
| Human prolactin | Peprotech. | 20 | ng/mL |
| (PRL) | Reference number: 100-07 | | |
| Human chorionic | Alpha Diagnostic Intl. | 1 | μg/mL |
| gonadotropin (hCG) | Catalog number: RP-1484 | | |
| Human placental | R&D. | 20 | ng/mL |
| lactogen (hPL) | Catalog number: 5757-PL/CF | | |
| ROCK inhibitor | Y-27632 | 10 | uM |
| (*) only added upon | | | |
| embryo attachment | | | |
| Glucose | Sigma Aldrich. | 7.5 | mM |
| | Reference number: G8644 | | |

| | | |
|---|---|---|
| Insulin-Transferrin- | Thermo Scientific. Gibco. | 1X |
| Selenium-Ethanolamin | Catalog number: 51500-056 | |
| (ITS-X) | | |
| Non-essential amino | Thermo Scientific. Gibco. | 1X |
| acids | Catalog number: 11140-035 | |
| Essential amino acids | Thermo Scientific. Gibco. | 1X |
| | Catalog number: 11130-036 | |

### REFERENCES

Burton, G. J., Cindrova-Davies, T., & Turco, M. Y. (2020). Review: Histotrophic nutrition and the placental-endometrial dialogue during human early pregnancy. Placenta, 102, 21-26. https://doi.org/10.1016/J.PLACENTA.2020.02.008
D'hauterive, S. P., Close, R., Gridelet, V., Mawet, M., Nisolle, M., & Geenen, V. (2022). Human Chorionic Gonadotropin and Early Embryogenesis: Review. International Journal of Molecular Sciences 2022, Vol. 23, Page 1380, 23(3), 1380. https://doi.org/10.3390/IJMS23031380
Gellersen, B. B., & Brosens, J. J. (2014). Cyclic decidualization of the human endometrium in reproductive health and failure. Endocrine Reviews, 35(6), 851-905. https://doi.org/10.1210/ER.2014-1045
Gellersen, B., Brosens, I. A., & Brosens, J. J. (2007). Decidualization of the human endometrium: Mechanisms, functions, and clinical perspectives. In Seminars in Reproductive Medicine (Vol. 25, Issue 6, pp. 445-453). © Thieme Medical Publishers. https://doi.org/10.1055/s-2007-991042
Nikas, G., & Makrigiannakis, A. (2003). Endometrial Pinopodes and Uterine Receptivity. Annals of the New York Academy of Sciences, 997(1), 120-123. https://doi.org/10.1196/ANNALS.1290.042
Pérez-Deben, S., Gonzalez-Martin, R., Palomar, A., Quiñonero, A., Salsano, S., & Dominguez, F. (2020). Copper and lead exposures disturb reproductive features of primary endometrial stromal and epithelial cells. Reproductive Toxicology, 93 (January), 106-117. https://doi.org/10.1016/j.reprotox.2020.01.008
Turco, M. Y., Gardner, L., Hughes, J., Cindrova-Davies, T., Gomez, M. J., Farrell, L., Hollinshead, M., Marsh, S. G. E., Brosens, J. J., Critchley, H. O., Simons, B. D., Hemberger, M., Koo, B.-K., Moffett, A., & Burton, G. J. (2017). Long-term, hormone-responsive organoid cultures of human endometrium in a chemically defined medium. NATURE CELL BIOLOGY, 19 (5). https://doi.org/10.1038/ncb3516

1. A method of generating an in vitro endometrium assembloid, said method comprising the steps of:
(i) growing endometrial stromal cells inside a hydrogel matrix; and
(ii) layering cells and/or fragments from endometrial epithelial organoids on top of the cultured endometrial stromal cells in hydrogel matrix of step (i);
thereby generating a three-dimensional endometrium assembloid comprising a stromal compartment, an outer luminal epithelial layer and a functional secretory glandular endometrium,
wherein the hydrogel matrix comprises one or more of the following components: dextran polymers, cyclodextrin crosslinkers comprising matrix metalloprotease cleavable sites, collagen I, collagen III, collagen VI and fibronectin.

2. The method of claim 1, wherein step (i) comprises growing the endometrial stromal cells inside the hydrogel matrix for at least 4 days, and/or
wherein step (i) comprises growing the endometrial stromal cells inside the hydrogel matrix until the endometrial stromal cells exhibit one or more morphological properties characteristic of endometrial stromal cells in natural endometrium and/or the natural stromal layer of natural endometrium.

3. The method of claim 2, wherein the endometrial stromal cells are isolated from endometrial biopsies, and/or wherein the stromal cells are primary endometrial stromal cells, or
wherein the endometrial stromal cells are from an immortalised human endometrial stromal cell line.

4. The method of claim 3, wherein the endometrial epithelial cells are from endometrial biopsies, and/or wherein the epithelial cells are primary endometrial epithelial cells, or
wherein the endometrial epithelial cells are from an immortalised human endometrial epithelial cell line.

5. The method of claim 4, wherein the method additionally comprises the step of: (iii) growing the endometrial stromal cells inside a hydrogel matrix and layered cells and/or fragments from endometrial epithelial organoids for at least 3 days.

6. The method of claim 5, wherein the cells and/or fragments from endometrial epithelial cell organoids are from an epithelial organoid culture system comprising the steps of:
(i) resuspending tissue isolates enriched for epithelial cells, and/or glandular elements obtained from endometrial and/or decidual tissue, in a solubilised basement membrane preparation comprising laminin, collagen IV, heparin sulphate proteoglycans, entactin/nidogen and growth factors; and
(ii) culturing the resuspended tissue isolates of step (i) in a culture medium comprising epidermal growth factor (EGF), Noggin, R-spondin-1, nicotinamide, N-Acetyl-L-cysteine, hepatocyte growth factor (HGF), the Alk3/4/5 inhibitor A83-01 and fibroblast growth factor 10 (FGF10).

7. The method of claim 6, wherein:
the tissue isolates enriched for epithelial cells are obtained from endometrial and/or decidual tissue biopsies by enzymatic digestion to disaggregate stromal cells, wherein said enzymatic digestion is halted prior to disaggregation of epithelial cell-containing elements of the biopsy, and/or prior to disaggregation of glandular elements; and/or
the solubilised basement membrane preparation is extracted from Engelbreth-Holm-Swarm (EHS) mouse sarcoma.

8. (canceled)

9. The method of claim 1, wherein:
the cells and/or fragments of endometrial epithelial culture organoids retain the bi-potential capability to generate luminal and glandular lineages, and are able to self-organise into a superficial luminal epithelium continuous to glandular-like invaginations in culture; and/or
the stromal compartment permits penetration and invasion of an embryo in a physiological three-dimensional scaffold in vitro.

10. (canceled)

11. The method of claim 1, wherein the outer luminal epithelial layer comprises acetylated α-tubulin rich cells.

12. The method of claim 1, wherein the outer luminal epithelial layer acts as site of anchorage to support initial embryo apposition and adhesion in vitro.

13. The method of claim 1, wherein the functional secretory glandular endometrium provides histotrophic nourishment to support embryo growth during implantation in vitro.

14. The method of claim 1, wherein the endometrium assembloid undergoes proliferation in response to oestrogen.

15. The method of claim 1, wherein the endometrium assembloid undergoes:
decidualization of the stromal compartment, wherein decidualization comprises morphological transformation and reorganisation of the actin cytoskeleton in the cells of the stromal compartment, and/or secretion of prolactin and/or insulin-like growth factor binding protein 1 (IGFBP1) by the cells of the stromal compartment; and
differentiation of the epithelial endometrial layer in response to oestrogen and progesterone stimulation in culture, wherein differentiation comprises:
morphological remodelling of the cells of the outer epithelial layer, and/or secretion of glycodelin and/or uterine milk by the cells of the outer luminal epithelial layer and functional secretory glandular endometrium; or
formation of pinopodes on the apical surface of the outer luminal epithelial layer.

16. (canceled)

17. (canceled)

18. The method of claim 15, wherein the secretions are accessible to the embryo in vitro.

19. (canceled)

20. A three-dimensional endometrium assembloid obtainable by the method of claim 1.

21. An in vitro method of determining embryo implantation ability and/or window of receptibility to human embryo implantation, said method comprising the steps of:
(i) providing the three-dimensional endometrium assembloid of claim 20, wherein the endometrial stromal cells and endometrial epithelial cells have been isolated from an endometrial biopsy obtained from a subject;
(ii) stimulating the endometrium assembloid using one or more of oestrogen, progesterone and cyclic AMP (CAMP), thereby rendering the endometrium assembloid receptive to embryo implantation; and
(iii) culturing a human stem cell based embryo-like or human blastocyst donated by an IVF patient in the presence of the stimulated endometrium assembloid, thereby allowing the blastoid or blastocyst to implant into the endometrium assembloid,
optionally wherein the blastocyst is isolated from the subject or the blastoid is derived from cells of the subject.

22. A method of screening for an embryo implantation-modulating agent, said method comprising the steps of:
(i) providing the three-dimensional endometrium assembloid of claim 20;
(ii) stimulating the endometrium assembloid using one or more of oestrogen, progesterone and cyclic AMP (CAMP) in the presence and/or absence of a test agent; and
(iii) culturing a human blastoid or human blastocyst in the presence of the stimulated endometrium assembloid,
wherein a difference between the implantation of the blastoid or blastocyst into the endometrium assembloid stimulated in the presence of the test agent and the implantation of the blastoid or blastocyst into the endometrium assembloid stimulated in the absence of the test agent is indicative of the implantation-modulating effect of said agent.

23. The method of claim 21, wherein step (ii) comprises stimulating the endometrium assembloid using oestrogen for between 2 and 8 days, followed by stimulating the endometrium assembloid using oestrogen, progesterone and CAMP for between 2 and 10 days.

24. The method of claim 22, wherein step (iii) is performed between 1 and 20 days after stimulating step (ii).

25. A method of culturing human stem cell based embryo-like or human blastocysts, comprising sequentially:
(i) culturing human stem cell based embryo-like or human blastocysts for between 5 and 9 days post-fertilisation or equivalent in human embryo medium 1 (HEM-1);
(ii) culturing the human blastoids or human blastocysts from step (i) for between 9 and 12 days post-fertilisation or equivalent in human embryo medium 2 (HEM-2); and
(iii) culturing the human blastoids or human blastocysts from step (ii) beyond 12 days post-fertilisation or equivalent in human embryo medium 3 (HEM-3).
